(19) United States
(12) Patent Application Publication
(10) Pub. No.: US 2024/0180898 A1
(43) Pub. Date: Jun. 6, 2024

(54) USE OF AMINOTHIAZOLE DERIVATIVE IN TREATMENT OF IMMUNE INFLAMMATION

(71) Applicant: INNAMUNE PHARMACEUTICAL CO. LTD., NINGBO (CN)

(72) Inventors: PING ZHOU, NINGBO (CN); FENGCHAO JIANG, NINGBO (CN)

(21) Appl. No.: 18/430,780

(22) Filed: Feb. 2, 2024

Related U.S. Application Data
(63) Continuation of application No. PCT/CN2023/111113, filed on Aug. 3, 2023.
(63) Continuation of application No. PCT/CN2022/110395, filed on Aug. 4, 2022.

(30) Foreign Application Priority Data
Aug. 5, 2021 (CN) 202110897623.3

(51) Int. Cl. A61K 31/496; A61K 9/06; A61P 1/16; A61P 3/04; A61P 17/02; A61P 17/06; A61P 25/24; A61P 25/28; A61P 27/02; A61P 37/08

(52) U.S. Cl. CPC A61K 31/496; A61K 9/06; A61P 1/16; A61P 3/04; A61P 17/02; A61P 17/06; A61P 25/24; A61P 25/28; A61P 27/02; A61P 37/08

(57) ABSTRACT

The invention provides a use of aminothiazole derivative in treatment of immune inflammation, the aminothiazole derivative can treat surface immune inflammatory dermatitis and immune surface inflammation, such as psoriasis, neurodermatitis, specific dermatitis eczema, atopic dermatitis, urticaria, seborrheic dermatitis and alopecia, abnormal scar, pathological pigmentation, etc. There are also immune inflammation caused by chronic rhinitis, conjunctivitis, dry eye, allergic asthma, chronic bronchitis, mucosal and epidermal injuries; It also includes chronic refractory immune inflammatory diseases such as various autoimmune diseases such as diabetes, systemic lupus erythematosus, rheumatoid disease; Metabolic inflammatory diseases such as obesity, fatty liver, metabolic inflammatory syndrome; Inflammatory cancer such as chronic colitis colon cancer, chronic hepatitis cirrhosis liver cancer; Ischemia-reperfusion injury, graft rejection and graft-versus-host disease, chronic inflammation of the nervous system such as neurodegeneration, Alzheimer's syndrome, depression, etc., have obvious effects, safety and non-toxic.

### CROSS-REFERENCE TO RELATED APPLICATIONS

The present application is a continuation-application of International Patent Application (PCT) No. PCT/CN2022/110395 and No. PCT/CN2023/111113, PCT/CN2023/111113 filed on Aug. 3, 2023, which claims priority of PCT/CN2022/110395, and PCT/CN2022/110395 filed on Aug. 4, 2022, which claims foreign priority of Chinese Patent Application No. 202110897623.3, filed on Aug. 5, 2021, the present application also claims the entire contents of which are hereby incorporated by reference.

### TECHNICAL FIELD

The invention relates to the field of biomedicine, in particular to the use of aminothiazole derivative in treatment of immune inflammation.

### BACKGROUND

For a long time, it has been proven that the inhibitory regulation of the body's immune system is the key to the treatment of many diseases, such as the treatment of organ transplantation rejection, autoimmune diseases, inflammatory diseases, ischemia reperfusion injury and so on.

Chronic refractory inflammatory diseases can form a variety of inflammatory diseases in various organs, such as autoimmune diseases, various immune inflammatory diseases, chronic inflammatory diseases and so on. Superficial inflammation refers to the inflammation occurring in the skin or mucosal surface layer, including infectious inflammation and immune inflammation caused by immune disorders, which often develop into chronic refractory inflammation. Immune surface inflammation is a major category of refractory immune inflammatory diseases. Due to the characteristics of the surface tissue, it is easy to have a large number of inflammatory irritants and a large number of inflammatory cells. In the process of cell pyroptosis, the inflammation continues to expand, and a vicious cycle makes the surface inflammation persist. Immune surface inflammation, including dermatitis and mucosal inflammation, such as dermatitis, rhinitis, conjunctivitis, mucosal and epidermal injury inflammation, and so on. Chronic vicious cycle immune response is considered to be a key step in the occurrence and development of refractory surface inflammation. During the occurrence and development of refractory surface dermatitis, the activation of immune cells such as macrophages, neutrophils and mast cells rich in the surface tissue will lead to the synthesis and secretion of various inflammatory cytokines, attracting more inflammatory cells to gather in the surface tissue. Triggering a series of vicious cycles is a chronic immune response, which is the main reason for the persistence of superficial inflammation. Therefore, inhibiting inflammation and interrupting the vicious cycle has become the key to the treatment of immune inflammatory surface inflammation, but it is not limited to the above inflammation, such as autoimmune diseases, various immune inflammatory diseases, and chronic inflammatory diseases also have a similar vicious cycle.

Pyroptosis is a new inflammatory mechanism, which plays an important role in various kinds of inflammation. During pyroptosis, cell membrane is damaged, cell swelling breaks, and a large number of inflammatory factors are released, leading to severe local inflammatory response, and a large number of inflammatory cells are raised and aggregated locally, forming a vicious cycle of inflammation. According to the classical pyroptosis pathway, pathogenic stimuli such as LPS and HMGB1 can bind to RAGE receptors, enter cells through pinocytosis, and activate Pro-Caspase-11, leading to pyroptosis (see FIG. 1).

In the prior art, an aminothiazole derivative, whose structure and function are disclosed in the Chinese Invention patent CN201110049687.4 “2-aminothiazole derivative and its Preparation Method and Application”. This small molecule compound blocks the activation of NF-κB by inhibiting myeloid differentiation protein (MYD-88). The resulting immunosuppressive effect, anti-transplant rejection, anti-autoimmune disease, anti-ischemia-reperfusion injury & anti-chronic inflammatory reaction, anti-endotoxemia, and other aspects of treatment.

However, our experiments found that other NF-κB inhibitors did not play a good role in chronic refractory inflammatory diseases, and there were no commercially available or clinically targeted inflammatory drugs targeting NF-κB. Therefore, this study conducted further research on the process of pyroptosis, and found a new effective pathway of this aminothiazole derivative in the inhibition of pyroptosis.

### SUMMARY

The purpose of the invention is to provide the use of the aminothiazole derivative in the preparation of immune inflammatory drugs. Due to its strong inhibiting effect on pyroptosis, the aminothiazole derivative of the invention can not only effectively inhibit inflammation, but also block the vicious cycle of inflammation by inhibiting the assembly and activation of inflammatory bodies and the occurrence of pyroptosis of innate immune cells (mainly macrophages). It has achieved good therapeutic effect in a series of pharmacodynamic trials of immunoinflammatory surface inflammation and immunoinflammatory diseases in various organs and tissues.

The molecular structure of the aminothiazole derivative described in the present invention is as follows:

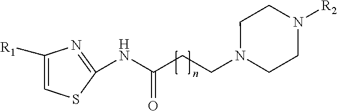

wherein, R1 is phenyl group or substituted aromatic group; R2 is benzyl, substituted benzyl, phenyl, or substituted phenyl; n=0, 1, 2.

Our mechanism study confirmed for the first time another pyroptosis activation pathway, from the classical TLR/MyD88 activation pathway, activation of NF-κB, and then through the NLRP3-Caspase-1-GSDMD-N pathway to activate pyroptosis. Further experiments showed that these aminothiazole derivatives could inhibit the expression of NLRP3 in immune cells, undergo secondary inhibition of Caspase-1, and lead to the inhibition of pyroptosis. It can also inhibit the release of inflammatory factors IL-1b and IL-18 by reducing the pathway of Pro-IL-1b and Pro-IL-18 in cells. (See Embodiment 2 for relevant experiments)

Thus, these aminothiazole derivatives can be used as pyroptosis inhibitors, NLRP3 inflammasome inhibitors, Caspase-1/Caspase-11 inhibitors, Gasdermin D (GSDMD-N, the N-terminus of GSDMD) inhibitors.

The aminothiazole derivatives of the invention have very small molecules, stable structures, can easily penetrate cell membranes, and can be used both in vivo and in vitro. These compounds can also be used to treat chronic, refractory, immunoinflammatory diseases such as autoimmune diseases (e.g. diabetes, systemic lupus erythematosus), various immune chronic inflammatory diseases (e.g. chronic colitis, allergies), ischemia-reperfusion injury, chronic inflammation of the nervous system (e.g. neurodegeneration, Alzheimer's syndrome, depression, etc.), and so on.

The aminothiazole derivatives such as 2-(4-(4-methylphenyl) piperazine-1-yl) -N-(4-phenylthiazole-2-yl) acetamide (product No. INNA1602, from Innamune Pharmaceutical Co. Ltd., the following product number is also from the company); 2-(4-phenylpiperazine-1-yl)-N-(4-phenylthiazole-2-yl) acetamide (product No. INNA1603); 3-(4-(4-methoxy phenyl) piperazine-1-base)-N-(4-phenyl thiazole-2-) propionamide (product No. INNA1604); 3-(4-benzylpiperazine-1-yl)-N-(4-phenylthiazole-2-yl) propionamide (product No. INNA1605), 2-(4-benzylpiperazine-1-yl)-N-(4-phenylthiazole-2-yl) acetamide (product No. INNA1608); 3-(4-benzylpiperazine-1-yl)-N-(4-(3-methoxyphenyl)thiazole-2-yl) propionamide (product No. INNA1609); 3-(4-(3-methyl phenyl)piperazine-1-base-N-(4-(3-methyl phenyl)thiazole-2-base) propionamide (product No. INNA1611); 2-(4-benzyl-base)piperazine-1-N-(4-(3-methyl phenyl)thiazole-2-base) acetamide (product No. INNA1612); 3-(4-benzyl-base)piperazine-1-N-(4-(3-methyl phenyl)thiazole-2-base) No. propionamide (product INNA1613); 3-(4-benzyl-base)piperazine-1-N-(4-(4-methyl phenyl)thiazole-2-base) propionamide (product No. INNA1614); 3-(4-(3-methoxy phenyl)piperazine- 1-base)-N-(4-phenyl thiazole-2-) propionamide (product No. INNA1615); 2-(4-(4-methoxy phenyl)piperazine-1-base)-N-(4-phenyl thiazole-2-) acetamide (product No. INNA1616); 2-(4-benzylpiperazine-1-yl)-N-(4-(4-methylphenyl)thiazole-2-yl) acetamide (product No. INNA1617) . They have the same efficacy, stability, and safety verified by the inventor. The structural formula is as follows:

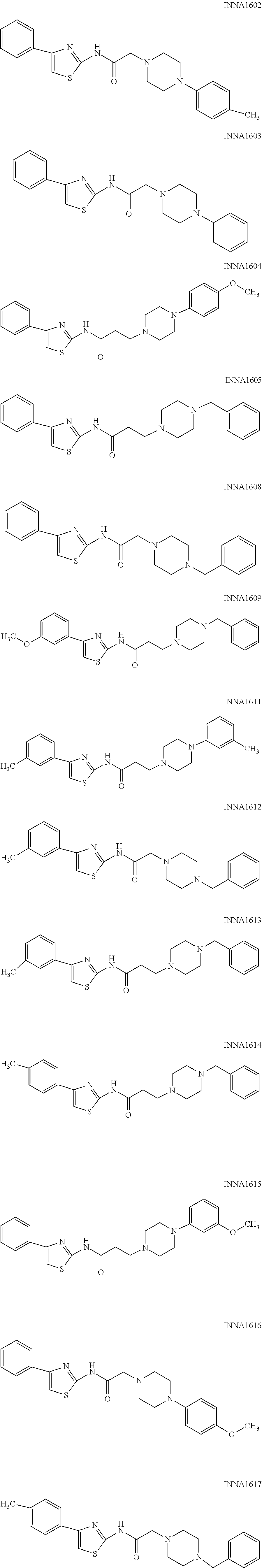

Previous studies have shown thatthe aminothiazole derivatives (a series of small molecule compounds) can effectively inhibit the synthesis and secretion of inflammatory factors, and the curative effects are basically the same. The present invention mainly takes INNA1605 and non-infectious surface dermatitis as an example to illustrate.

The present invention also discloses the aminothiazole derivatives as external preparations (ointments, liniment, spray, patch, drops) of the specific example, and internal preparations (oral and intravenous agents) used for the surface immune inflammation and other refractory autoimmune disease. For example, it is used in a variety of immunoinflammatory dermatitis (psoriasis, neurodermatitis, specific dermatitis eczema, atopic dermatitis, urticaria, and similar dermatitis).

### THE BENEFICIAL EFFECTS OF THE INVENTION

the exccellent efficacy of the aminothiazole derivatives in many immune-inflammatory diseases has been fully confirmed in animal experiments and compassionate treatment of patients, including: 1. Surface immune inflammatory dermatitis such as psoriasis, neurodermatitis, specific dermatitis eczema, atopic dermatitis, urticaria, seborrheic dermatitis, hair loss, abnormal scar formation, pathological pigmentation and other aspects have obvious effects, and are safe and non-toxic. There are other types of immune surface inflammation as follows but not limited to: chronic rhinitis, conjunctivitis, dry eye, allergic asthma, chronic bronchitis, immune inflammation caused by various mucosal and epidermal injuries, chronic inflammation of various mucosa and epidermis. 2, immune inflammatory diseases also include other chronic, refractory, immune inflammatory diseases such as autoimmune diseases (eg. diabetes, systemic lupus erythematosus), various immune chronic inflammatory diseases and inflammatory carcinogenesis (such as chronic colitis colon cancer, chronic hepatitis cirrhosis liver cancer, etc.), allergies, ischemia-reperfusion injury, metabolic inflammatory diseases (such as obesity, fat Liver, metabolic inflammatory syndrome, etc.), graft rejection and graft-versus-host disease, chronic inflammation of the nervous system (such as neurodegeneration, Alzheimer's syndrome, depression, etc.), and so on.

### BRIEF DESCRIPTION OF THE DRAWINGS

FIG. 1 shows the mechanism of macrophage activation pyroptosis.

FIG. 2 shows that this series of aminothiazole derivatives ((INNA1602 (TJ-2), INNA1605 (TJ-5), INNA1608 (TJ-8), INNA1609 (TJ-9), INNA1611 (TJ-11), INNA16012 (TJ-12)) can inhibit the secretion of a large number of inflammatory factors from macrophages induced by LPS, and has the same inhibitory effect on inflammatory factors.

FIG. 3 shows INNA1605 (TJ-5) inhibits pyroptosis of bone marrow derived macrophages. A and B. Cell apoptosis (PI+ flow staining) of bone marrow derived macrophages in each group. C. LDH concentration of bone marroe derived macrophage supernatant in each group. D-F. Expression of GSDMD and GSDMD-N protein in bone marrow derived macrophages in each group.

FIG. 4 shows INNA1605 (TJ-5) inhibits classic and non classic pyroptosis pathway of bone marrow derived macrophages, and to reduce the synthesis and secretion of a variety of inflammatory cytokines. A-C. Expression of NLRP3 inflammasome and its component protein and non-classical pyroptosis pathway related protein in macrophages of bone marrow origin in each group. D. Synthesis and secretion of various inflammatory factors in bone marrow-derived macrophages in each group.

FIG. 5 shows the effect of using INNA1605 ointment to treat psoriasis in Balb/c mouse strains. a. Normal contrast group, b. IMQ model group, c. INNA1605 treatment group.

FIG. 6 shows the pathological changes in skin tissue after treating psoriasis with INNA1605.

FIG. 7 shows that INNA1605 can inhibit the MAPK signaling pathway in a psoriasis model.

FIG. 8 shows the immunohistochemical results of MPO (neutrophil) and CD86 (macrophage) in the skin tissue of psoriasis treated with INNA1605.

FIG. 9 shows that INNA1605 inhibits the infiltration and proliferation of mast cells in a psoriasis model. A. High magnification observation of H&E staining in mouse skin tissue. B. The staining results of C-kit (CD117), a specific indicator of mast cells in mouse skin tissue.

FIG. 10 shows that INNA1605 significantly reduces the expression of CD80.

FIG. 11 shows that INNA1605 inhibits the expression of intradermal lymphokines such as IL-1beta, IL-6, IL-17a, TNF-alpha, MyD88 mRNA, and Cxcl2 mRNA in psoriasis.

FIG. 12 shows the macroscopic and weight maps of the spleens in each group after treating psoriasis with INNA1605, as well as the flow cytometry and proportion statistics of T cells and their subsets derived from the spleens in each group.

FIG. 13 shows the H&E pathological map of keratinocytes in the skin tissue of psoriasis mice treated with INNA1605 to reduce excessive keratinization and proliferation of cells, as well as the measurement and statistics of keratinized skin thickness.

FIG. 14 shows the pathological changes in mice with atopic dermatitis (eczema) treated with INNA1605.

FIG. 15 shows the expression levels of inflammatory factors in the skin of mice with atopic dermatitis (eczema) treated with INNA1605.

FIG. 16 shows the changes in inflammatory factors in mice with atopic dermatitis (eczema) at different times: the models at 8, 10, and 12 days were tested, and IL-4 and IL-13 levels were elevated on the 8th day, reaching high levels from 10 to 12 days (expression levels were almost the same as the internal reference); The expression levels of TSLP and MyD88 peaked 8 days ago and decreased from 10 to 12 days.

FIG. 17 shows the inhibition of colonic mucosal inflammation and proliferation of colonic mucosal epithelial cells after INNA 1605 treatment. A. H&E pathological changes of the colon mucosa. B. The infiltration of neutrophils in the colon mucosa, namely the immunohistochemical pathological staining of MPO. C&D The BrdU staining and Ki-67 immunohistochemistry pathology of the colonic mucosa, reflect the proliferation of colonic mucosal epithelial cells in each group.

FIG. 18 shows the clinical study of INNA1605 ointment for treating mosquito bite allergic dermatitis (sympathetic treatment).

FIG. 19 shows the clinical study of INNA1605 ointment in the treatment of neurodermatitis (sympathetic treatment).

FIG. 20 shows the clinical study of INNA1605 ointment in the treatment of eczema (sympathetic treatment).

FIG. 21 shows the clinical study of INNA1605 ointment in the treatment of urticaria (sympathetic treatment).

FIG. 22 shows the clinical study of INNA1605 ointment in the treatment of psoriasis (sympathetic treatment).

FIG. 23 shows the clinical study of INNA1605 ointment in the treatment of proteinosis dermatitis (sympathetic treatment).

FIG. 24 shows that INNA1606 (TJ-M2010-6) can prevent the occurrence of type I diabetes in NOD mice, and can also treat type I diabetes that has already occurred if it is used in the early stage of the disease. The mechanism is that MyD88 inhibitors can significantly alleviate islet inflammation and rescue pancreatic cells.

FIG. 25 shows that INNA1605 (TJ-M2010-5) significantly reduced the weight of high-fat diet obese mice.

FIG. 26 shows that INNA1605 (TJ-M2010-5) significantly inhibits the activation of B cells (lupus like cells) and the differentiation of the B cells into plasma cells induced by R848.

FIG. 27 shows that INNA1605 (TJ-M2010-5) significantly inhibits the secretion of autoimmune antibodies and inflammatory factors by R848 induced B cells (lupus like cells).

FIG. 28 shows that INNA1605 (TJ-M2010-5) significantly alleviates secondary ischemia- reperfusion injury after acute cerebral infarction, resulting in an 80% reduction in the volume of brain tissue necrosis in acute cerebral infarction.

FIG. 29 shows INNA1605 (TJ-M2010-5) improving cognitive abilities in APP/PSI mice or mice with Hammer's syndrome (water maze test).

FIG. 30 shows that INNA1605 (TJ-M2010-5) significantly reduces Aß deposition in the brain of APP/PS1 model mice with Alzheimer's syndrome. Representative immunohistochemical images of the whole brain (A), cortex (B), and hippocampus (C) regions of mice in each group at 9 months of age and the percentage of Aβ patch area (D). (A bar=1000 μm; B-C bar=200 μm)(###p<0.001 vs

Control group; * p<0.05 vs APP/PSI group; N=3 control group; N=5 APP/PSI & APP/PS1+INNA1605 group)

FIG. 31 shows that INNA1605 (TJ-M2010-5) significantly inhibits the activation of microglia in the brain of APP/PSI mice, thereby reducing inflammation of neural tissue in the brain. Representative immunohistochemical images of the whole brain (A), cortex (B), and hippocampus (C) regions of each group of mice at 9 months of age, as well as the percentage of Iba-1 positive microglia (D). (A bar=1000 μm; B-C bar=200 μm) (#p<0.05, ###p<0.001 vs Control group; *p<0.05, **p<0.01 vs APP/PSI group; N=3 control group; N=5 APP/PS1 & APP/PS1+INNA1605 group).

FIG. 32 shows INNA1605 (TJ-M2010-5) preventing depressive behavior and inflammation in mice.

FIG. 33 shows the mechanism diagram of INNA1605 (TJ-M2010-5) preventing depression behavior and inflammation induced by COVID-19 spike protein (CSDS-/SARS-907 COV-2 Spike RBD).

FIG. 34 shows INNA1605 (TJ-M2010-5) successfully transplanted skin that could not be successfully transplanted (Balb/c mouse skin transplanted to B6 mice).

FIG. 35 shows that short-term postoperative medication of INNA1605 (TJ-M2010-5) induced permanent immune tolerance in mouse heart transplantation. A. After allogeneic heart transplantation (Balb/c mouse heart transplantation to B6 mice), TJ-M2010-5 was administered for two weeks, and 60% of the heart transplants were not rejected. B. Long-term surviving recipient mice received a second co donor mouse heart transplant (Balb/c mouse heart transplant to B6 long-term surviving mice), with 100% non rejection, confirming the development of immune tolerance.

FIG. 36 shows that INNA1605 (TJ-M2010-5) significantly inhibits the growth of liver cancer (subcutaneous implantation of H22 cancer cells).

FIG. 37 shows a comparison of the experimental effects of INNA1605 (TJ-M2010-5) on the prevention and treatment of obesity and fatty liver, in this figure, HFD is the high-fat diet group, TJ-5 is the INNA1605 or TJ-M2010-5 group.

FIG. 38 shows a comparison of tissue pathology testing using INNA1605 (TJ-M2010-5) for the prevention and treatment of obesity and fatty liver in experimental mice, in this figure, HFD is the high-fat diet group, TJ-5 is the INNA1605 or TJ-M2010-5 group.

FIG. 39 shows INNA1605 (TJ-5) inhibits suppress pyroptosis in mice model of hepatic IRI. A. Western blotting analysis showing proteins levels of GSDMD and cleaved GSDMD (GSDMD-N) in liver tissues. B and C. Western blotting results illustrating expression level of NLRP3, pro-caspase-1, cleaved caspase-1, pro-caspase-11, and cleaved caspase-11 in liver tissue samples.

FIG. 40 shows a comparison of the experimental effects of INNA1605 (TJ-5) on the prevention and treatment of dry eye, in this figure, EDE is the model of dry eye group. TJ-5 decreases corneal inflammation in the models of dry eye.

FIG. 41 shows a comparison of the experimental effects of INNA1605 (TJ-5) on the prevention and treatment of allergic rhinitis, in this figure, AD is the model of allergic rhinitis group, TJ-5 alleviates the inflammatory response of allergic rhinitis.

FIG. 42 shows a comparison of the experimental effects of INNA1605 (TJ-5) on the wound healing and scarring after burn and skin trauma. in this figure, Vehicle is the model of skin wound group, TJ-5 accelerates wound healing and prevents skin scar formation.

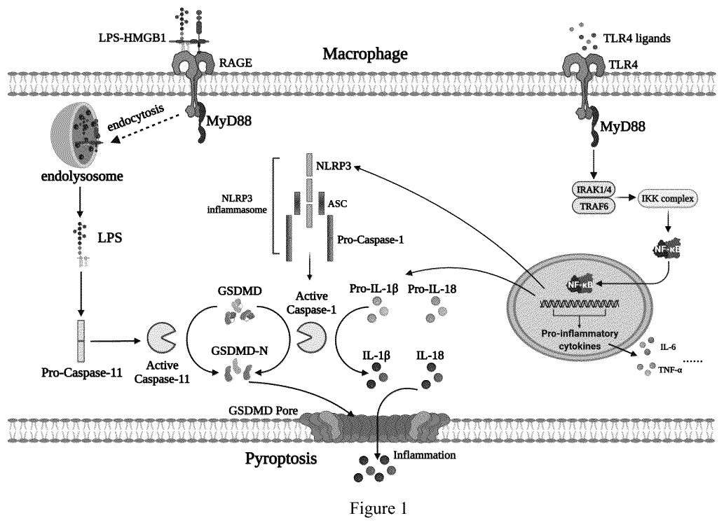

### DETAILED DESCRIPTION OF THE EMBODIMENTS

The following will provide a detailed description of the present invention in conjunction with the accompanying drawings and specific embodiments. The materials used in the embodiments can be obtained through commercial channels.

### Embodyment 1: Small Molecule Series of Compounds of the Aminothiazole Derivatives

[INNA1602 (TJ-M2010-2, TJ-2), INNA1605 (TJ-M2010-5, TJ-5), INNA1608 (TJ-M2010-8, TJ-8), INNA1609 (TJ-M2010-9, TJ-9) INNA1611 (TJ-M2010-11, TJ-11), INNA16012 (TJ-M2010-12, TJ-12)] can inhibit the secretion of inflammatory factors from macrophages induced by LPS.

macrophage grouping (in vitro):

Cultivate macrophages derived from mouse bone marrow in vitro.

The experiment was divided into 8 groups, namely:
Control group (untreated mouse bone marrow-derived macrophages),
LPS group (500 ng/ml LPS stimulated macrophages for 24 hours),
LPS+TJ-2 group (2 hours before LPS stimulation, administer 30 umol/L TJ-2 drug stimulation),
LPS+TJ-5 group (2 hours before LPS stimulation, administer 30 umol/L TJ-5 drug stimulation),
LPS+TJ-8 group (2 hours before LPS stimulation, administer 30 umol/L TJ-8 drug stimulation),
LPS+TJ-9 group (2 hours before LPS stimulation, administer 30 umol/L TJ-9 drug stimulation),
LPS+TJ-11 group (2 hours before LPS stimulation, administer 30 umol/L TJ-11 drug stimulation),
and LPS+TJ-12 group (2 hours before LPS stimulation, administer 30 umol/L TJ-12 drug stimulation).

After 24 hours of LPS stimulation, the cell culture supernatant was removed and ELISA was performed to detect the concentration of various inflammatory factors in the supernatant.

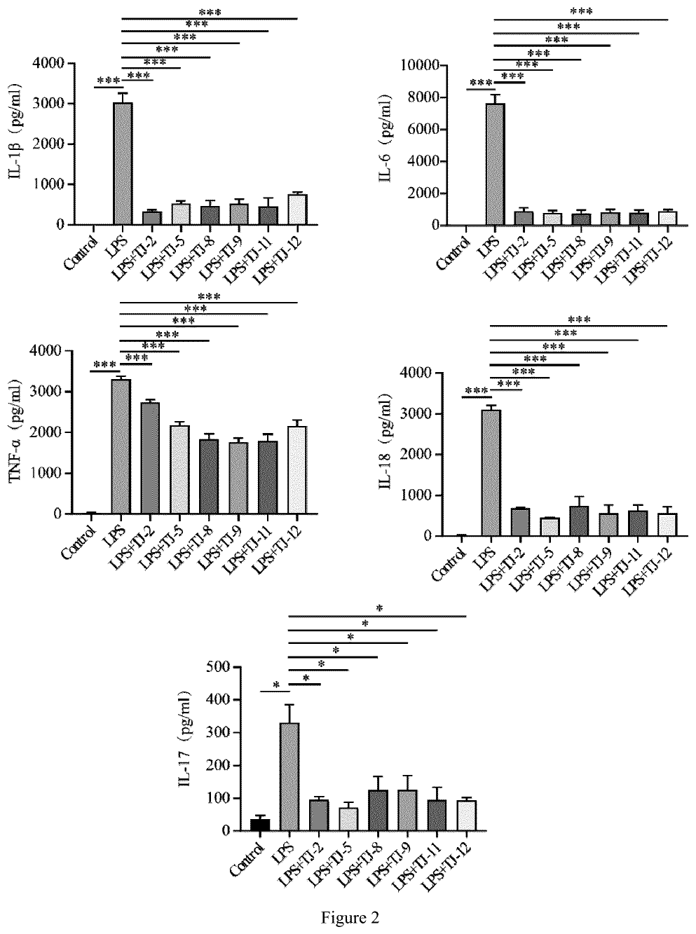

The experimental results obtained are shown in FIG. 2, which shows that all compounds of aminothiazole derivatives [INNA1602 (TJ-2), INNA1605 (TJ-5), INNA1608 (TJ-8), INNA1609 (TJ-9), INNA1611 (TJ-11), INNA16012 (TJ-12)] can inhibit the secretion of various inflammatory factors in macrophages, such as IL-1β, Il-6, TNF-α, IL-18, IL-17; and INNA1602 (TJ-2),INNA1605 (TJ-5), INNA1608 (TJ-8), INNA1609 (TJ-9), INNA1611 (TJ-11), and INNA16012 (TJ-12) have the same inhibitory effect on inflammatory factors.

### Embodyment 2: INNA1605 (TJ-M2010-5, TJ-5) Inhibits Cell Pyroptosis of Bone Marrow Derived Macrophages in Vitro

Preparation and grouping of bone marrow derived macrophage pyroptosis model:

Model preparation: After LPS (500 ng/ml) stimulation of bone marrow derived macrophages for 6 hours, ATP (5 mmol/L) stimulation for 2 hours, followed by cell collection and supernatant detection.

The experiment was divided into four groups, namely:
Control group (normal cell culture medium),
Model group (with LPS and ATP stimulation added to the culture medium),
TJ-5 (10 μmol/L) group (2 hours before LPS stimulation, administer 10 μmol/L TJ-5 drug stimulation),
TJ-5 (30 μmol/L) group (2 hours before LPS stimulation, administer 30 μmol/L TJ-5 drug stimulation).

Collect cells and their supernatant for detection at the endpoint of the experiment.

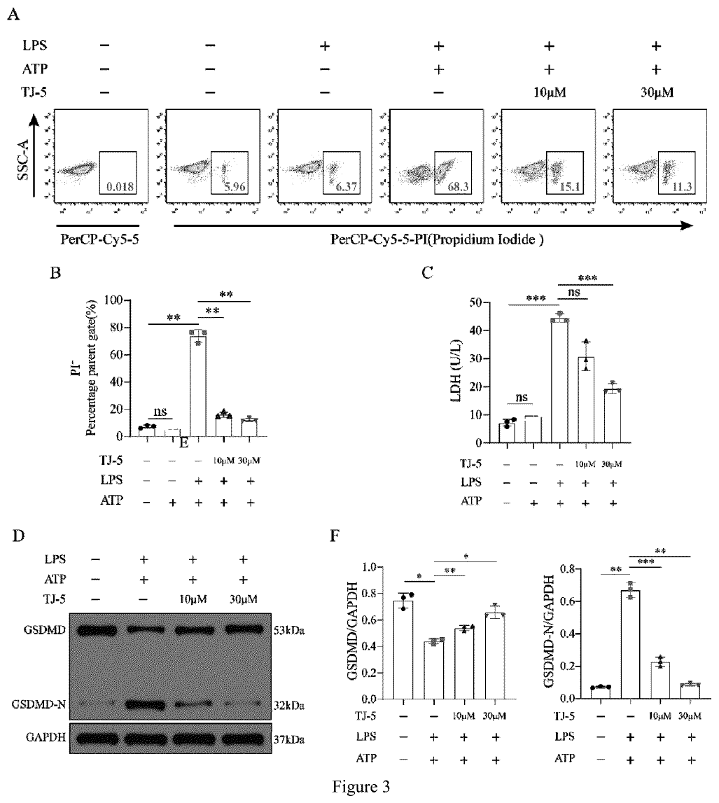

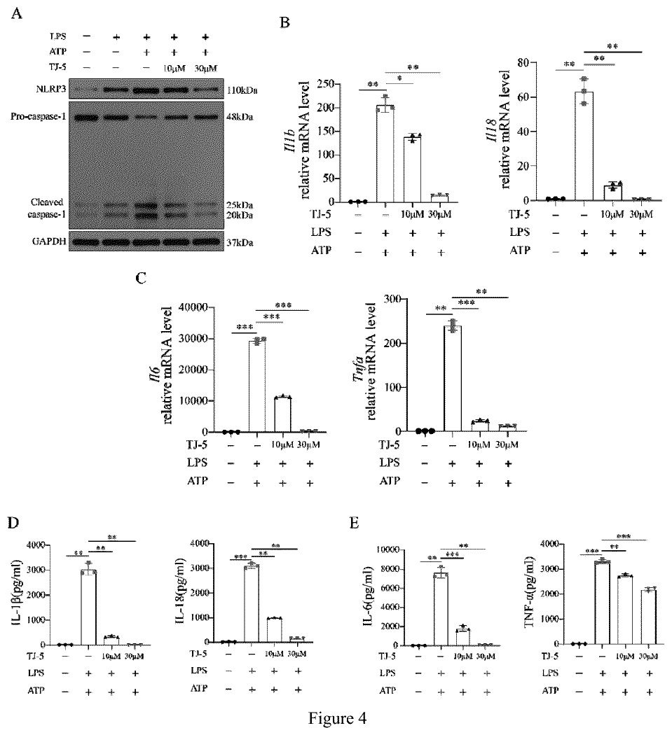

The experimental results obtained (see FIG. 3) show that after LPS and ATP stimulation, the PI+cells of bone marrow derived macrophages significantly increased, and the secretion of LDH in the cell culture supernatant also increased significantly. INNA1605 (TJ-M2010-5, TJ-5) can inhibit the apoptosis of bone marrow derived macrophages induced by LPS and ATP, and reduce the secretion of LDH (FIG. 2C, 2D), indicating that INNA1605 can inhibit the death of bone marrow derived macrophages induced by LPS and ATP. INNA1605 significantly inhibits the activation and cleavage of GSDMD in bone marrow derived macrophages induced by LPS and ATP, indicating that INNA1605 can inhibit pyroptosis of bone marrow derived macrophages (FIG. 3E, 3F). Moreover, INNA1605 can block the assembly and activation of NLRP3 inflammasomes, inhibit both classical and non classical cell pyroptosis pathways (FIG. 4A-C), thereby inhibiting the maturation and release of Pro- IL-1b and Pro-IL-18 in bone marrow derived macrophages, disrupting the cascade reaction of inflammation, and reducing the secretion of various inflammatory factors (FIG. 4D-G)

### Embodyment 3: INNA1605 (TJ-M2010-5, TJ-5) is Used to Treat Psoriasis in Mice (Balb/c)

Preparation and grouping of mouse psoriasis model:

Model preparation: Male Balb/c mice aged 6-8 weeks were shaved on the back skin and coated with 5% imiquimod (IMQ) cream once a day for 7 consecutive days.

The experiment was divided into three groups, namely:
Psoriasis control group (without any treatment, psoriasis model group), TJ-M2010-5 systemic medication group (intraperitoneal injection), and TJ-M2010-5 local medication group (ointment group). The specific handling method is as follows:
TJ-M2010-5 systemic medication group: A Balb/c mouse psoriasis model was taken and TJ- M2010-5 was injected intraperitoneally at a dose of 50 mg/kg, once a day, for 2 consecutive weeks.
TJ-M2010-5 local medication group: A Balb/c mouse psoriasis model was taken, and 1% TJ-M2010-5 ointment was applied on the skin, once a day, for 2 consecutive weeks.

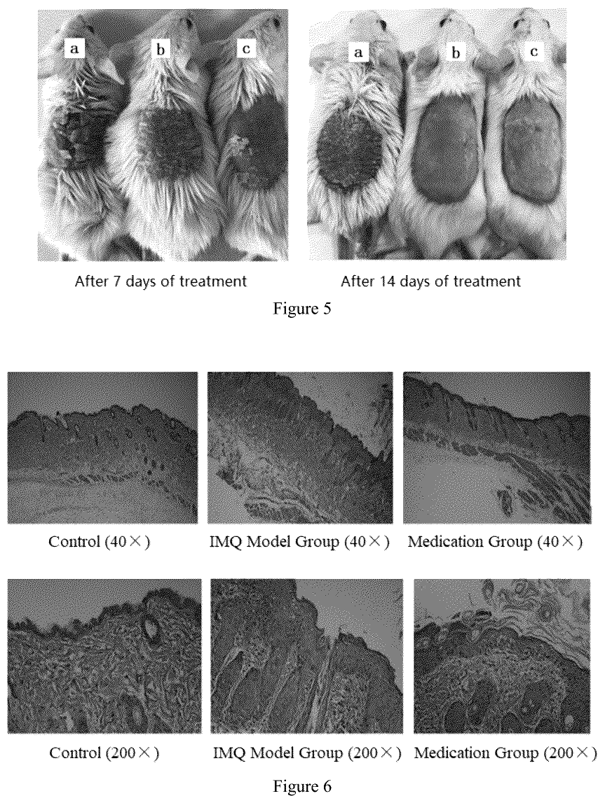

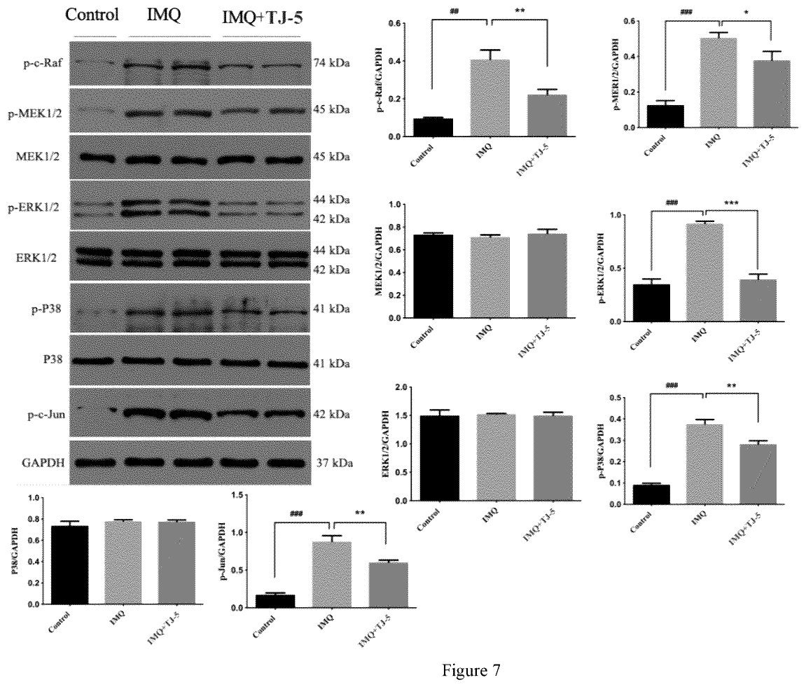

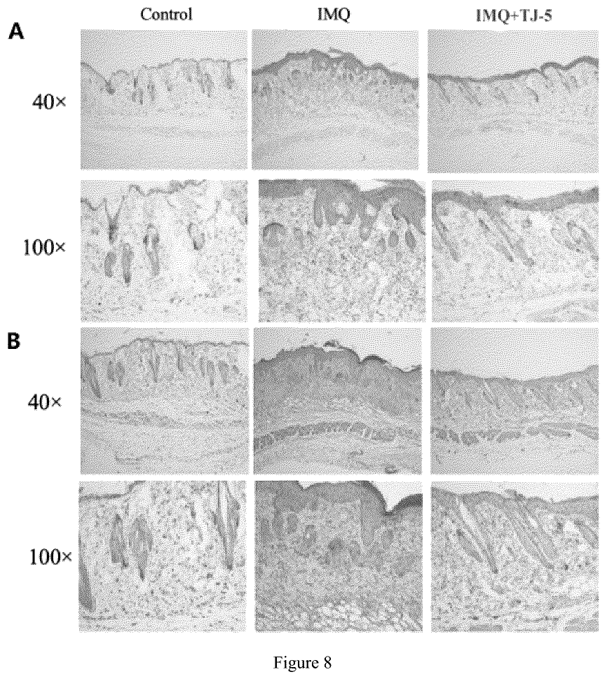

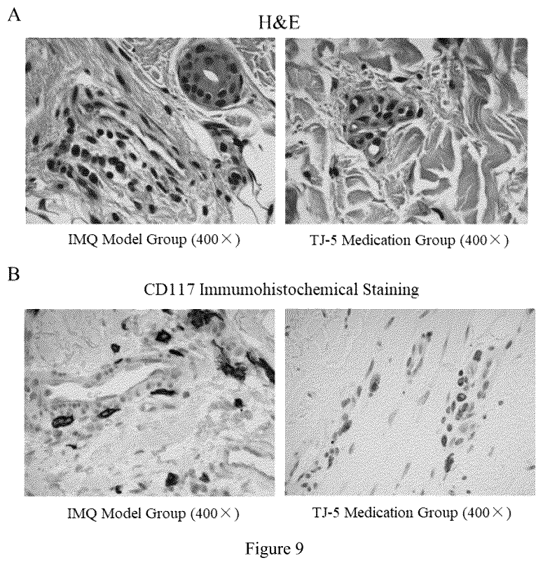

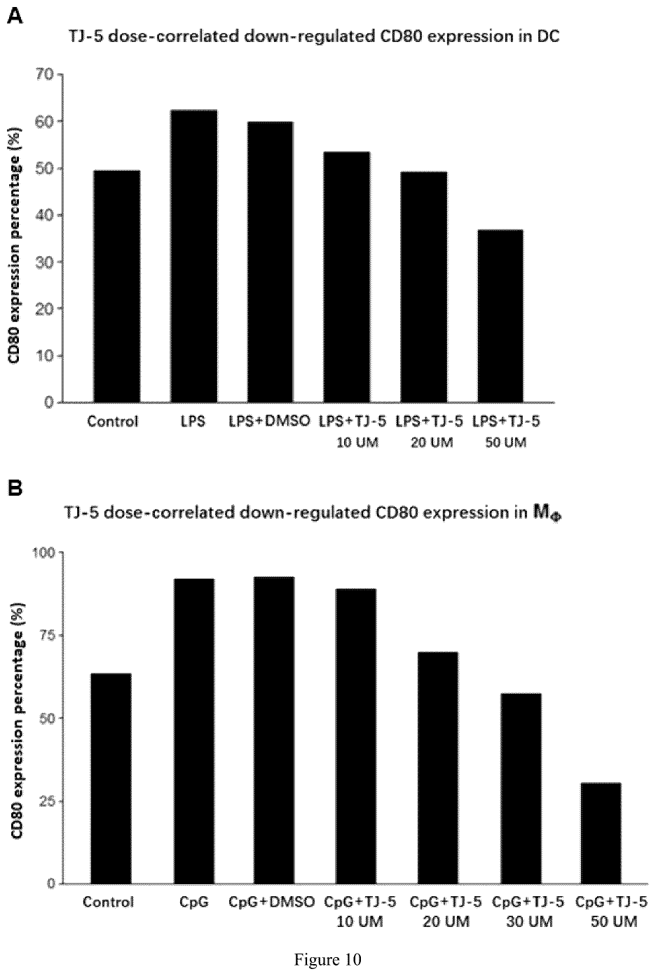

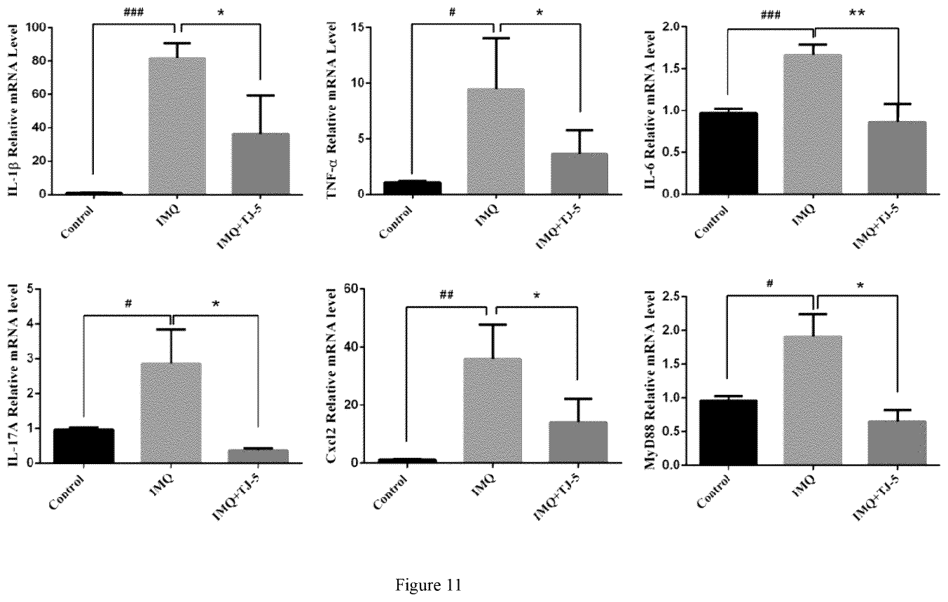

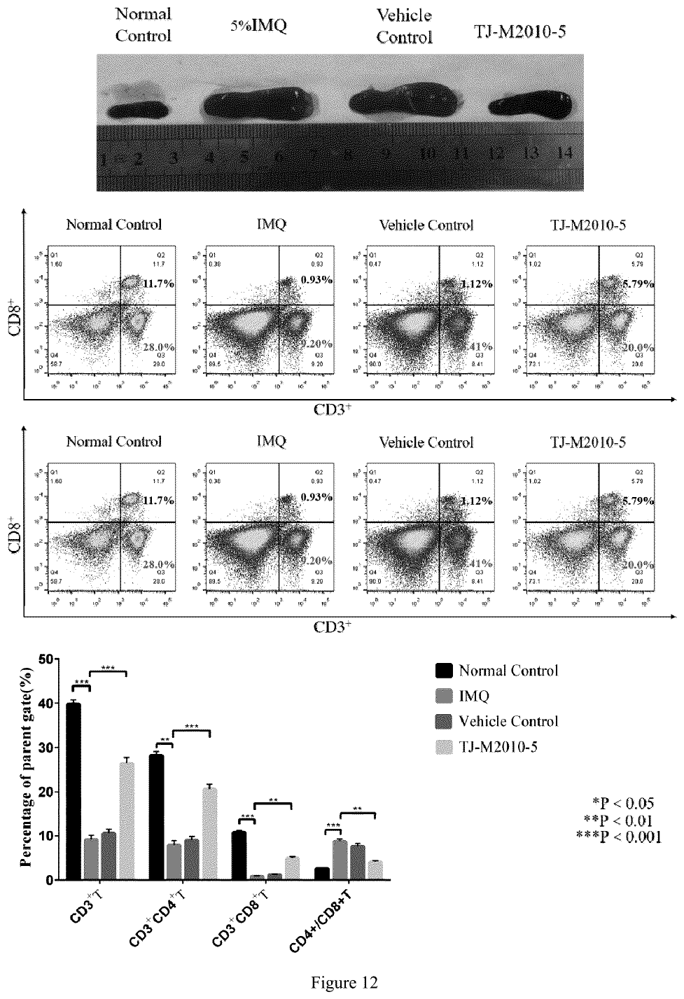

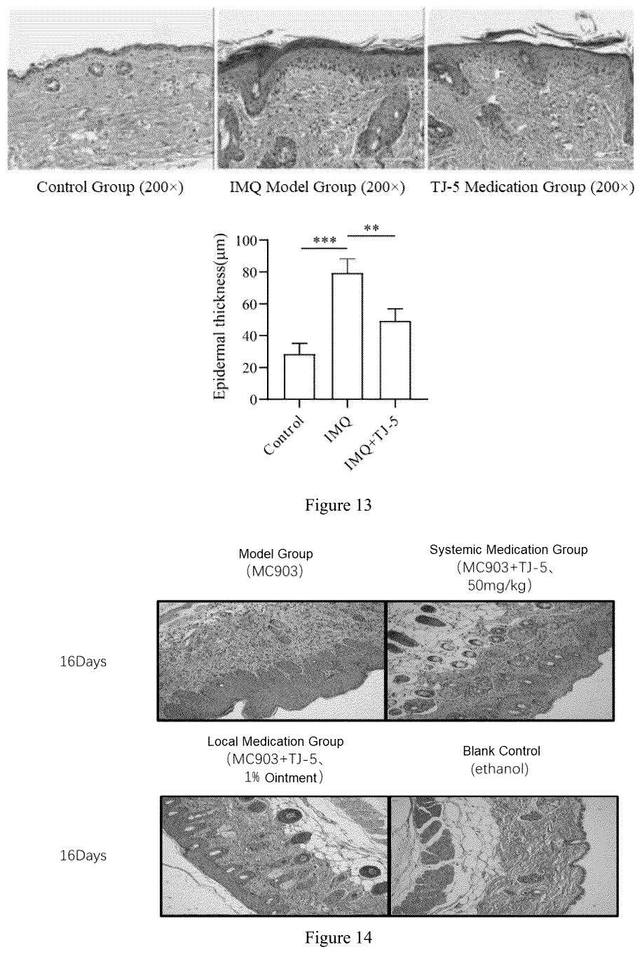

As shown in the experimental results graph (see FIG. 5), the results showed that both systemic and local medication had excellent therapeutic effects, with a significant improvement after 7 days and a cure effect achieved after 14 days. Two treatments can significantly reduce inflammatory cells in the skin (see FIG. 6), and the abnormally thickened stratum corneum in psoriasis can basically restore normal tissue structure after treatment (see FIG. 6). According to the in vitro study of Embodiment 2, INNA1605 treatment may inhibit the assembly and activation of NLRP3 inflammasomes in a psoriasis model, and inhibit the maturation and release of IL-18 and Pro-IL-1β, ultimately reduce the pyroptosis of skin tissue cells (see Embodiment 2). The reduction of cell pyroptosis directly interrupts the explosive vicious cycle of skin inflammation, and INN1605 simultaneously inhibits the MAPK signaling pathway (see FIG. 7). The direct result of both is a reduction in immune cell infiltration and activation, including macrophages, neutrophils, and mast cells (see FIGS. 8-10), further inhibiting the synthesis and secretion of inflammatory factors (see FIG. 11); The reduction of innate immune cell infiltration inevitably leads to the inhibition of antigen presentation to adaptive immune cells. Therefore, INN1605 can improve splenomegaly in psoriasis patients and correct the imbalance of spleen T cells and their subsets (see FIG. 12); At the same time, INN1605 can also inhibit excessive keratinization and proliferation of skin keratinocytes, resulting in a significant thinning of skin thickness. (See FIG. 13).

### Embodyment 4: INNA1605 (TJ-M2010-5, TJ-5) is Used to Treat Atopic Dermatitis/Eczema in Mice (C57)

Preparation and grouping of mouse psoriasis model:

Model preparation: Male C57 mice aged 6-8 weeks were treated with 2% M7309 (carboplatin) ointment on their ears once a day for 10 consecutive days.

The experiment was divided into three groups, namely:
Eczema control group (without any treatment, eczema model group), TJ-M2010-5 systemic medication group (intraperitoneal injection), and TJ-M2010-5 local medication group (ointment group). The specific handling method is as follows:
TJ-M2010-5 systemic medication group: A mouse eczema model was taken and TJ-M2010-5 was injected intraperitoneally at a dose of 50 mg/kg, once a day, for 2 consecutive weeks.
TJ-M2010-5 local medication group: Take a mouse eczema model, apply 1% TJ-M2010-5 ointment on the skin, once a day, for 2 consecutive weeks.

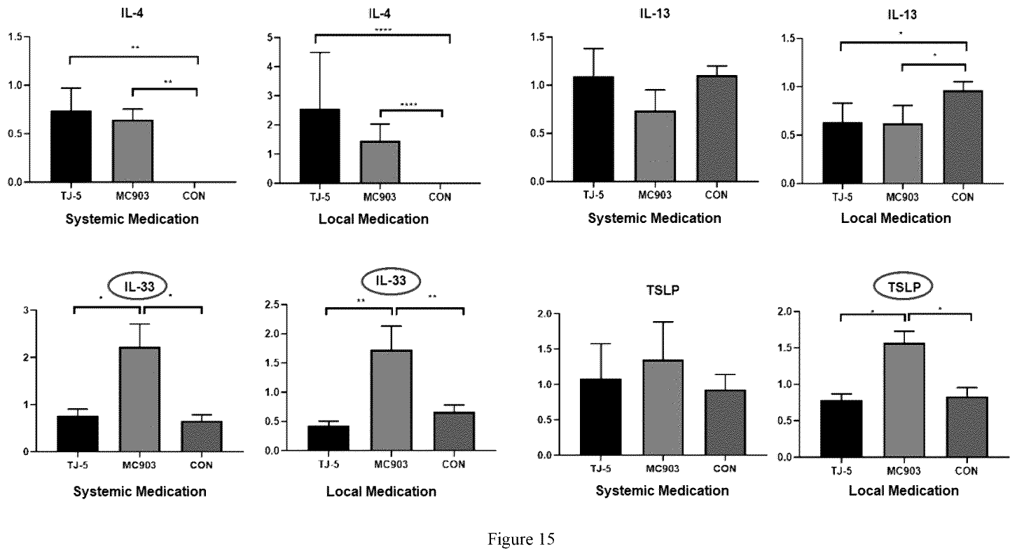

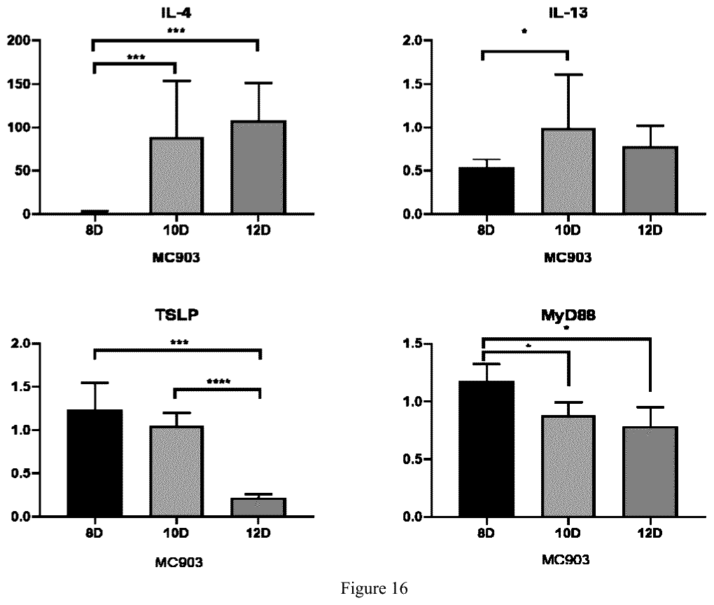

The experimental results obtained are shown in FIG. 14, which show that local medication has excellent therapeutic effects. After treatment, the abnormally thickened stratum corneum in dermatitis can basically restore normal tissue structure, and inflammation is significantly reduced (see FIG. 13). Various inflammatory factors decreased (see FIG. 15), and over time, the changes in various inflammatory factors showed peaks and decreases (see FIG. 16).

### Embodyment 5: INNA1605 (TJ-M2010-5, TJ-5) is Used to Treat Azomethane/Sodium Dextran Sulfate (AOM/DSS) Induced Inflammatory Colon Cancer in Mice (C57)

Preparation and grouping of mouse inflammatory colon cancer model:

Model preparation: 6-8-week-old male C57 mice were intraperitoneally injected with AOM (10 mg/Kg)/DSS (2.5%) to induce an inflammatory colon cancer model.

The experiment was divided into two groups, namely:
Inflammatory colon cancer model group (without any treatment, i.e. AOM/DSS group), TJ-M2010-5 medication group (intraperitoneal injection). The specific handling method is as follows:
TJ-M2010-5 medication group: Two days before inducing inflammatory colon cancer in C57 mice by intraperitoneal injection of AOM (10 mg/kg)/DSS (2.5%), TJ-M2010-5 was injected intraperitoneally at a dose of 50 mg/kg once a day for 10 consecutive weeks.

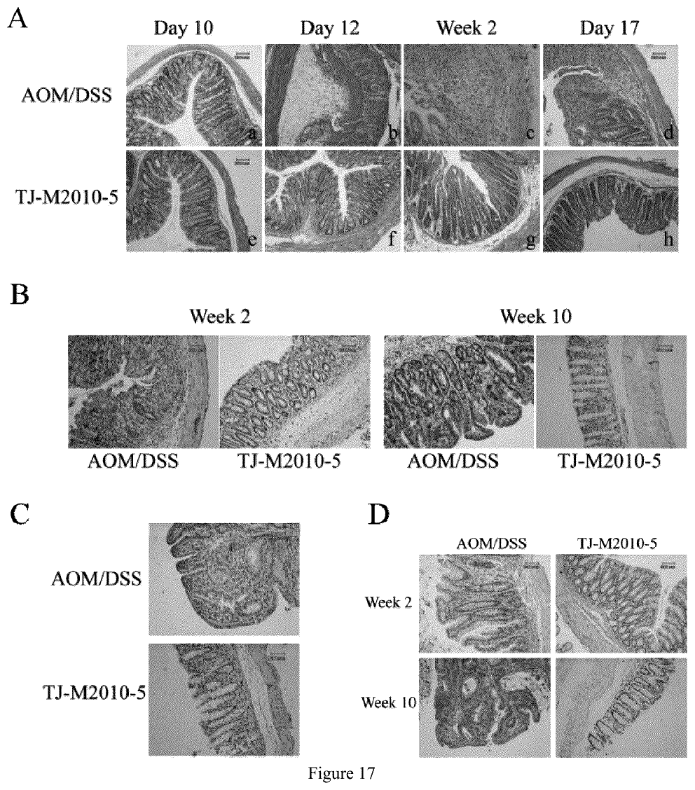

As shown in the experimental results graph (see FIG. 17), the model group had severe ulcerative colitis and 100% had a cancerous transformation. The results showed that after treatment with TJ-M2010-5, the therapeutic effect was excellent. After treatment, the inflammatory tissue damage of the colon mucosa in mice was significantly reduced (FIG. 17A), neutrophil infiltration was significantly reduced (FIG. 17B), and colitis disappeared. Moreover, after treatment with TJ-M2010-5, the proliferation of epithelial cells in the colon mucosa of mice was significantly inhibited, mucosal thickening was improved, and 100% no carcinogenesis was observed.

### Embodyment 6: Clinical study of 1% INNA1605 Ointment Applied to Sympathetic Treatment of Dermatitis Cases, Treating Atopic Dermatitis/Eczema, Neurodermatitis, Allergic Dermatitis, Urticaria, Proteinosis Dermatitis, etc.

Safety assessment: The preclinical trial results showed that INNA1605 has low toxicity to the body. Direct intravenous injection of 30 mg/kg or more will cause elevated liver enzyme toxicity, and it has no toxic effect at 10 mg/kg. When applying skin ointments to the maximum body area, only a trace drug concentration of 2-3 ng/ml can be measured in the blood, indicating that the amount absorbed through the skin is negligible and can be ignored, without toxic side effects on the body.

Sympathetic treatment: For patients with long-term recurrent attacks, ineffective treatment, and abnormal pain, sympathetic treatment is carried out under their active and strong demands, after passing the safety assessment by the physician, and under close observation.

Sympathetic treatment clinical studies: As these cases are only based on clinical studies and observations under safe and compassionate treatment, they cannot be used for NMPA registration and declaration, but only as research data for the purpose of evaluating the efficacy of the ointment.

### 6.1) 1% INNA1605 Ointment for Treatment of Mosquito Bite Allergic Dermatitis (Sympathetic Treatment Clinical Study)

Diagnosis: Allergic dermatitis and ulceration caused by mosquito bites

Joined on May 12, 2019

Treatment process: A 34-year-old male with dermatitis caused by mosquito bites, long-term itching and ulceration. No other medication was used. After using 1% INNA1605 cream for half a month, he recovered and stopped taking the medication.

Side effects: None

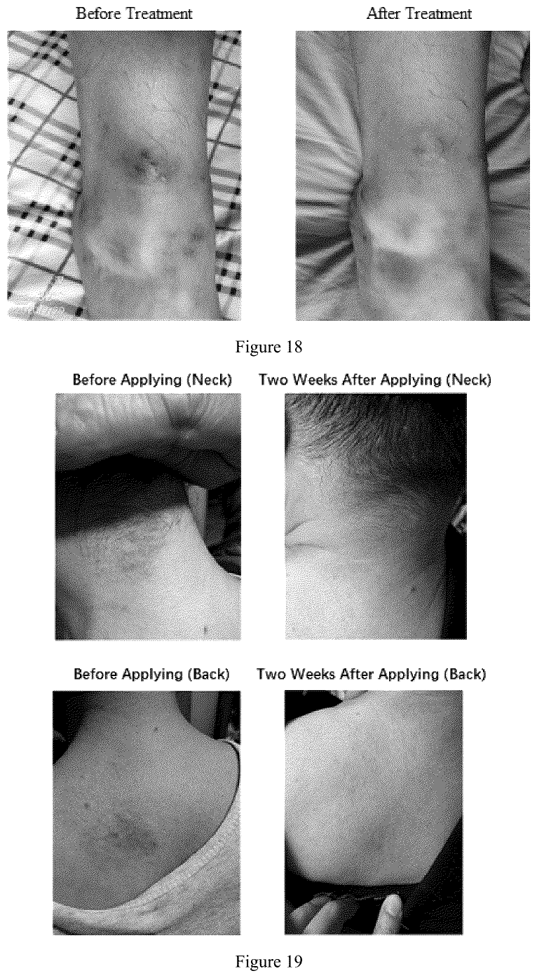

Therapeutic evaluation: Excellent, cured (see FIG. 18).

### 6.2) 1% INNA1605 Ointment for the Treatment of Neurodermatitis (Sympathetic Treatment Clinical Study)

Diagnosis: Neurodermatitis (multiple)

Joined on Oct. 20, 2019

Treatment process: A 58-year-old female, neurodermatitis of the posterior hairline, neck, and scapula, characterized by redness, swelling, and itching, is ineffective with other medications. After 3 weeks of using 1% INNA1605 cream, the redness and swelling subsided, the itching disappeared, and the skin returned to a normal state. It has completely recovered and has not recurred to this day.

Side effects: None.

Therapeutic evaluation: Excellent, cured (see FIG. 19).

### 6.3) 1% INNA1605 Ointment for the Treatment of Eczema (Sympathetic Treatment Clinical Study)

Diagnosis: Atopic dermatitis (eczema) (multiple)

Joined on May 25, 2020

Treatment process: A 30-year-old female with multiple dermatitis/eczema on both lower limbs, which has not healed for many years, severe itching, persistent scratching, and scabbing. Long term use of other medications, including hormone ointments, is ineffective and causes recurrent attacks. After two weeks of using INNA1605 (1%) cream on the affected area after enrollment, multiple areas of the legs no longer itched, and the deposition of pigmentation faded. The skin texture basically returned to normal, and there has been no recurrence since discontinuing the medication.

Patient's self description: The itching relieving effect is immediate, and after treatment, the scars are reduced and there is a whitening effect.

Side effects: None.

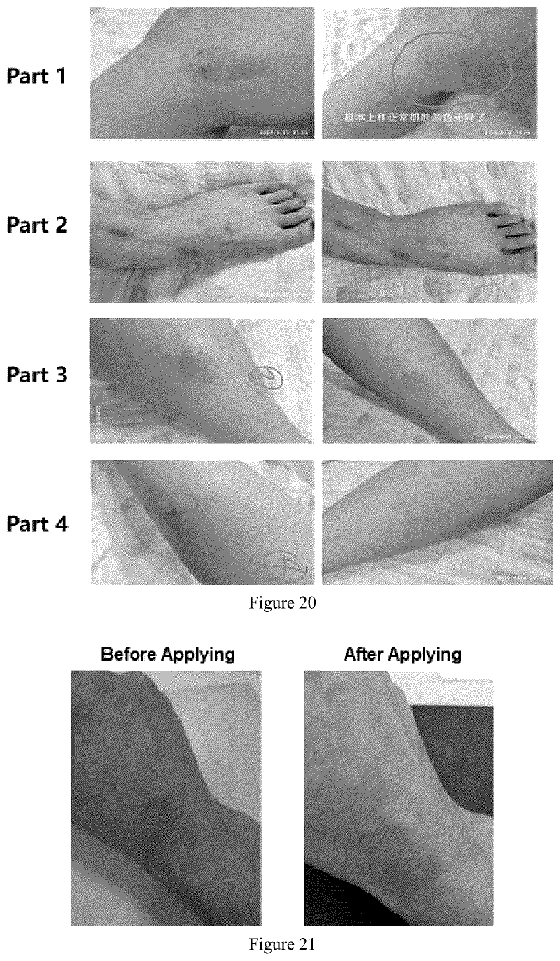

Therapeutic evaluation: Excellent, cured (see FIG. 20).

### 6.4) 1% INNA1605 Ointment for the Treatment of Urticaria (Sympathetic Treatment Clinical Study)

Diagnosis: Urticaria (multiple)

Joined on Sep. 7, 2020

Treatment process: A 54-year-old male. The patient has multiple urticaria throughout the body, which recur and cause severe itching, redness, and swelling. It is difficult to control scratching until it ruptures. Long term use of various other medications, including hormone ointments, has been ineffective, and recurrent episodes have persisted for many years. Previous use of Benvemod cream has not only been ineffective, but has also resulted in “heart pain” side effects.

After using 1% INNA1605 cream for 12 days after enrollment, the application site no longer had itching, no ulceration, and the redness and swelling significantly decreased. It returned to normal skin texture, completely recovered, and there has been no recurrence so far.

Side effects: None.

Therapeutic evaluation: Excellent, cured (see FIG. 21).

### 6.5) 1% INNA1605 Ointment for the Treatment of Psoriasis

Diagnosis: Severe psoriasis.

Joined on Sep. 17, 2019

Treatment process: A 69-year-old male, suffering from severe leg psoriasis for more than ten years, long-term treatment has been ineffective, including various hormone ointments and other treatments, which recur and cannot be cured. After using INNA1605 cream for only two weeks after enrollment, the itching disappeared, the surface scales disappeared, and the overall appearance became smooth to the naked eye. The brownish red patches became lighter in color, and the redness and swelling subsided. The patient felt good and had no side effects. However, due to the limited amount of ointment prepared at that time, there was no more ointment after only 2 weeks, and the medication was forced to be interrupted (the standard course of treatment should be more than 3 months). Subsequent patients lost contact and did not continue treatment.

Side effects: None.

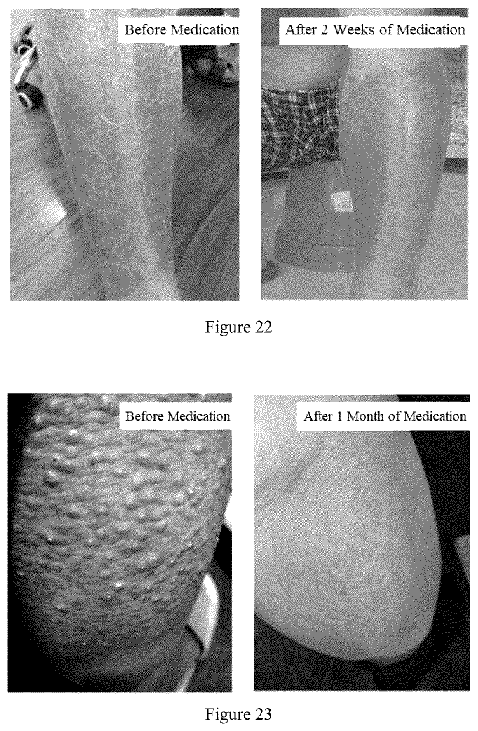

Efficacy evaluation: Excellent (see FIG. 22).

### 6.6) 1% INNA1605 Ointment for the Treatment of Proteinosis Dermatitis

Diagnosis: Protein deposition dermatitis.

Joined on Mar. 20, 2021

Treatment process: A 60-year-old male, protein deposition dermatitis has been present for decades, with itching, hyperplasia, and extremely unsightly appearance, without any medication treatment.

After enrollment, the use of INNA1605 (1%) cream can quickly relieve itching and reduce dermatitis. After one month of treatment, it has significantly improved and is currently continuing treatment. Treatment is required for more than three months.

Side effects: None.

Efficacy evaluation: Excellent (see FIG. 23).

The above experimental results directly demonstrate that the amino thiazole small molecule compound INNA1605 ointment has significant therapeutic effects in the treatment of immune inflammatory dermatitis such as psoriasis, atopic dermatitis (eczema), allergic dermatitis, neurodermatitis, urticaria, etc., and can achieve a complete cure. It will be a broad-spectrum and effective immunoinflammatory dermatitis ointment, which can almost fill the market gap for many difficult to treat skin diseases.

### Embodyment 7: Preparation of 2-Aminothiazole Derivative Cream Formulation

Taking 1% INNA1605 cream as an example, 1.1 Matrix preparation:

Oil phase preparation: Add 10 ml (8.7 g) of liquid paraffin, 7.0 g of stearic acid, 4.0 g of white vaseline, and 3.0 g of octadecanol to a beaker, stir and heat to about 70° C. until the oil phase is dissolved. Keep warm and set aside for later use.

Preparation of aqueous phase: 0.1 g of hydroxyethyl ester, 0.2 g of sodium dodecyl sulfonate, 0.40 ml (0.4 g) of triethanolamine, 10.0 ml of water, and 1.0 ml (1.0 g) of VE were added to a beaker, heated to 70° C., stirred until dissolved in aqueous phase, and kept warm for later use.

Preparation of essence phase: glycerin 12 ml (15.2 g), essence 0.2 ml, azone 0.5 ml (0.4 g), mix and stir evenly to obtain essence phase.

Substrate preparation process: add the aqueous phase to 49.8 ml of water at 50° C. under stirring, slowly add the oil phase to the above aqueous solution under stirring, continue to stir and emulsify for 3 h, add the essence phase after cooling to room temperature, continue to stir for 30 min tolhour until the mixed milky white semi coagulated solid is well mixed, and then solidify to obtain the matrix for standby.

### 1% INNA1605 Cream Preparation (Grinding Method)

Place 1.0 g of finely ground (sieved through a 100 mesh sieve) INNA1605 compound in a mortar, and add it to the prepared matrix in equal amounts and grind evenly. The preparation method for other INNA1605 cream formulations with varying concentrations is the same as above, but the drug content can be changed (such as adding 2 g of INNA1605 compound to the corresponding matrix according to the above method for a 2% INNA1605 cream formulation).

The preparation method for cream formulations of other 2-aminothiazole compounds such as INNA1602, INNA1603, INNA1604, INNA1605, INNA1608, INNA1609, INNA1611, INNA1612, INNA1613, INNA1614, INNA1615, INNA1616, and INNA1617 are the same as above.

### Embodyment 8: Preparation of 2-Aminothiazole Derivative Ointment Formulation

Taking the preparation of 1% INNA1602 ointment as an example

Preparation method: finely grind the drug through a 100 mesh sieve for later use; Heat and melt lanolin (9.0%), vaseline (85.0%), and stearic acid (4.5%) to 50° C. Add azone (transdermal absorption promoter, 1.5%), stir well, and cool to room temperature for later use. Add the mixture into the drug paste in equal increments, grinding it while adding until it cools down to room temperature.

The preparation method for ointment of other 2-aminothiazole compounds such as INNA1602, INNA1603, INNA1604, INNA1605, INNA1608, INNA1609, INNA1611, INNA1612, INNA1613, INNA1614, INNA1615, INNA1616, and INNA1617 are the same as above.

### Embodyment 9: INNA1606 (TJ-M2010-6, TJ-6) is Used to Treat Type I Diabetes in Mice

Preparation and grouping of mouse type I diabetes model:

Model preparation: purchase NOD mice from Beijing Weitong Lihua Biotechnology Co., Ltd. NOD mice will spontaneously develop autoimmune diabetes (type I diabetes).

The experiment is divided into two groups, namely:

NOD control group (without any treatment, model group);

INNA1606 (TJ-M2010-6, TJ-6) prophylactic drug group (orally administered by gavage): NOD mice were given TJ-M2010-6 (50 mg/kg) by gavage daily, from 8 weeks to 30 weeks, or until the onset of diabetes. The control group mice were simultaneously given the same dose of drug solvent;

INNA1606 (TJ-M2010-6, TJ-6) immediate treatment group (oral administration by gavage): two doses of TJ-M2010-6 were administered immediately on the first day when NOD mice were diagnosed with diabetes, and then twice a day (50 mg/kg) during the following four weeks of observation; The NOD control group was also given an equal amount of drug solvent;

INNA1606 (TJ-M2010-6, TJ-6) delayed treatment group (oral administration by gavage): the treatment dose and duration were the same as those of the immediate treatment group, while the NOD mice were given TJ-M2010-6 one week after the onset of diabetes, The NOD control group was also given an equal amount of drug solvent;

All NOD mice in the immediate treatment group and the delayed treatment group were observed 4 weeks after the onset of diabetes.

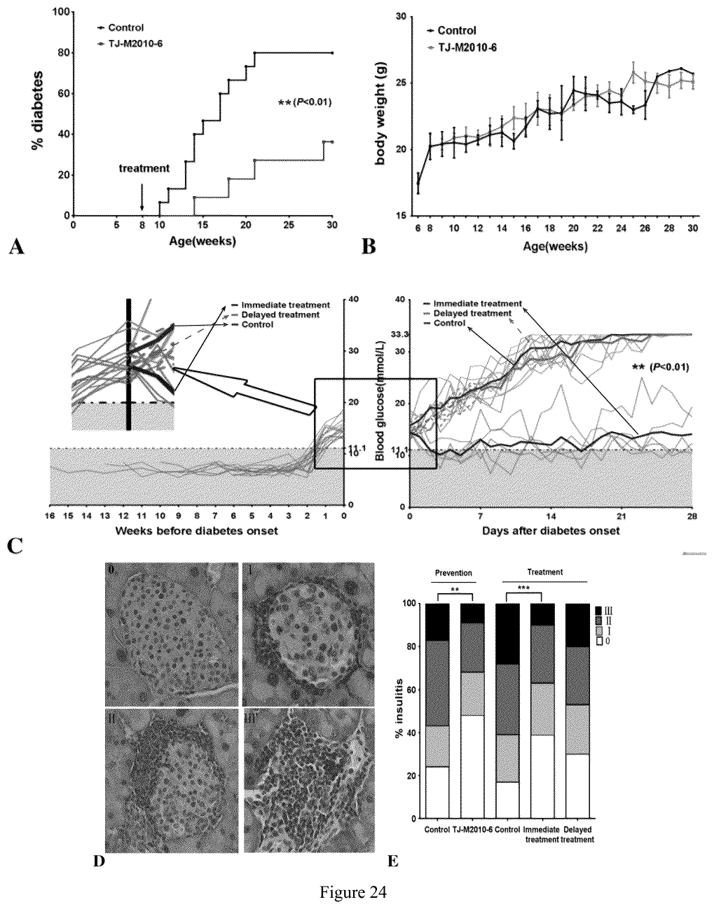

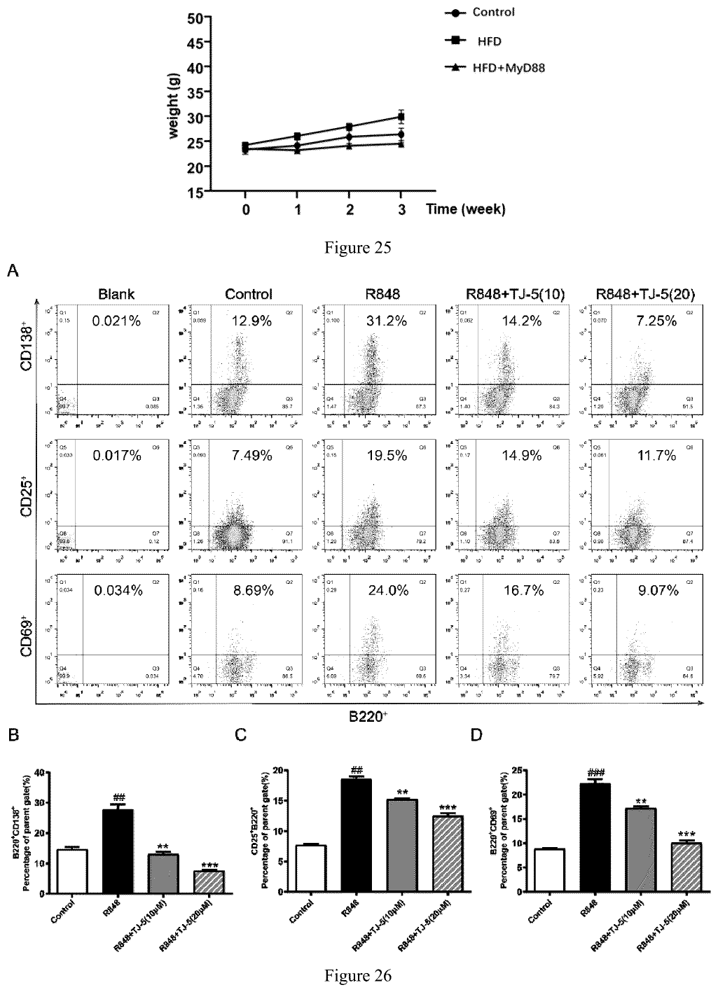

The experimental results obtained are shown in FIG. 24. The results show that INNA1606 (TJ-M2010-6, TJ-6) significantly reduces the proportion of mice still diagnosed with type I diabetes at the end of the experiment after treatment (FIG. 24A). Moreover, immediate treatment and delayed treatment have effects on type I diabetes in mice (FIG. 24C). INNA1606 plays a therapeutic role in type I diabetes by inhibiting islet inflammation (FIG. 24D, E). At the same time, INNA1606 can significantly reduce the weight of obese mice fed with high-fat diet (FIG. 25), which also indicates its potential to treat diabetes.

### Embodyment 10: INNA1605 (TJ-M2010-5, TJ-5) Can Correct Immune Disorders Induced by R848 in Lupus Like B Cells

Preparation and grouping of an in vitro lupus like B cell immune disorder model:

Model preparation: Mouse spleen derived B cells were purified using a magnetic bead sorting kit and cultured in vitro. Stimulating factor CD40L was added to maintain the long-term survival of B cells in vitro. Lupus like B cell immune disorders were induced by stimulating B cells with R848 (500 ng/ml) for 48 hours.

The experiment is divided into four groups, namely:
Control group (without any treatment, normal cultured B cells);
R848 group (model group): Stimulate B cells with R848 (500 ng/ml) for 48 hours;
TJ-5 (10 μM) Group: First use TJ-5 (10 μmol/L) Pre stimulate B cells for 3 hours, then stimulate B cells with R848 (500 ng/ml) for 48 hours;
TJ-5 (20 μM) Group: First use TJ-5 (20 μmol/L) Pre stimulate B cells with mol/L for 3 hours, then stimulate B cells with R848 (500 ng/ml) for 48 hours.

Collect cells and cell culture supernatant for detection after R848 stimulation is completed.

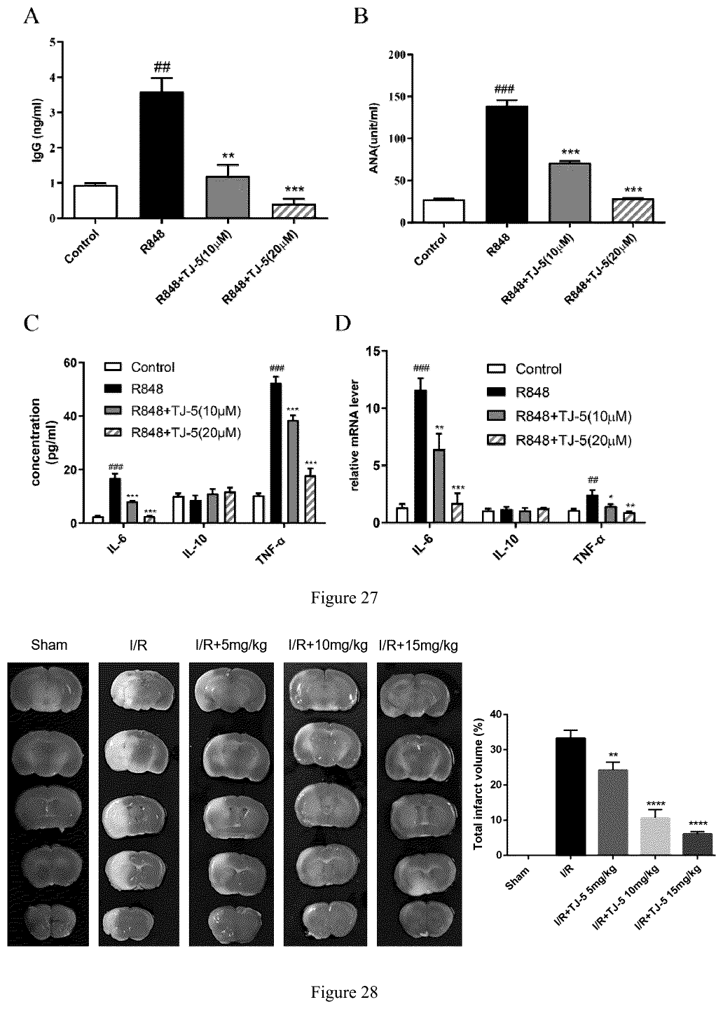

The experimental results obtained (see FIG. 26/27) show that R848 stimulation of B cells can cause B cell activation and differentiation into plasma cells, producing a large number of autoimmune antibodies and synthesizing and secreting a large number of inflammatory factors; INNA1605 (TJ-M2010-5, TJ-5) can inhibit abnormal activation and differentiation of B cells (see FIG. 26), and inhibit the secretion of B cell autoantibodies and inflammatory factors (see FIG. 27). INNA1605 can correct a series of immune disorders induced by R848 in lupus like B cells, and has the potential to treat systemic lupus erythematosus.

### Embodyment 11: INNA1605 (TJ-M2010-5, TJ-5) has a Significant Therapeutic Effect on Reperfusion Injury After Acute Cerebral Infarction

Preparation and grouping of a mouse model of reperfusion injury after acute cerebral infarction:

Model preparation: Adult male C57BL/6 mice (8-10 weeks old, weighing 22-25 g) were fasted for 8 hours before surgery and allowed to drink freely. Intraperitoneal injection of 1% pentobarbital sodium solution for anesthesia.

Middle cerebral artery occlusion (MCAO) model: Fix the mouse in a supine position, keep the mouse's head tilted to its left, spray disinfectant alcohol on the central area of the neck, scrape off the neck hair with a blade, vertically cut open the middle right position of the neck, bluntly separate the skin and thyroid, be careful not to damage the vagus nerve and parathyroid gland, and carefully separate and expose the common carotid artery (CCA), Gradually separate and expose the right external carotid artery (ECA) and internal carotid artery (ICA) upwards, arterial clamp closure of CCA and ICA, silk thread ligation of the end of ECA away from the heart, with silk thread on the end of ECA close to the heart, make a small incision in the ECA using precision ophthalmic scissors, slowly insert a thread plug coated with silicone into the ICA, ligate and fix the thread, and release the arterial clamp, continue to slowly insert the thread plug, and stop insertion when there is some resistance, At the same time, the Doppler blood flow meter monitored a significant decrease in cerebral blood flow in the ischemic side of the middle cerebral artery (MCA) area. After 1 hour of ischemia, the thread plug was slowly removed, the ECA was ligated, the neck skin was sutured, and placed in a 32° C. incubator. After the mice woke up, 1 ml of physiological saline was injected intraperitoneally for fluid replacement. The surgical steps of the sham group were basically the same as those of the model group, but did not block MCA. After the animal wakes up, it returns to its cage and eats freely.

The experiment is divided into four groups, namely:
Sham surgery group (Sham group): the surgical steps and postoperative treatment were the same as the model group, but did not block MCA;
Model group (I/R group): Establish an MCAO model, operate as described above, without special handling;
Vehicle control group (Vehicle group): After removing the thread plug, immediately inject an equal amount of physiological saline intravenously
Medication group (I/R+INNA1605 15 mg/kg group): After removing the thread plug, immediately inject 150 ul 2.5 mg/ml of INNA1605 solution (15 mg/kg) intravenously.

Mouse brain tissue was removed for pathological examination and subsequent molecular biology analysis after 1 hour of ischemia followed by 24 hours of reperfusion.

The experimental results obtained are shown in FIG. 28. The results show that INNA1605 (TJ-M2010-5, TJ-5) significantly reduces the infarct volume of acute cerebral infarction in mice, with a staggering reduction of 80%. It can be said that INNA1605 (TJ-M2010-5, TJ-5) has great potential for treating acute cerebral infarction after reperfusion injury.

### Embodyment 12: Therapeutic Effect of INNA1605 (TJ-M2010-5, TJ-5) on Alzheimer's Disease

Preparation and grouping of Alzheimer's disease mouse models:

Model preparation: APP/PSI transgenic mice were purchased from Shanghai Slake Experimental Animal Co., Ltd. The transgenic mice spontaneously developed Alzheimer's disease at 8 to 9 months of age and did not require external treatment.

The experiment is divided into three groups, namely:

Drug group (APP/PS1+INNA1605 group): APP/PSI transgenic mice were given INNA1605 at a dose of 50 mg/Kg/day (twice a day, 25 mg/Kg each time, intraperitoneal injection once, gavage once);

Model group (APP/PSI group): APP/PSI transgenic mice were given an equal dose of solvent (ddH2O);

Control group: C57BL/6 mice were given an equal dose of solvent (ddH2O).

At 9 months of age, Morris water maze test was conducted to evaluate the spatial learning and memory abilities of APP/PSI mice. After the behavioral experiment was completed, pentobarbital sodium (100 mg/kg) was injected into the abdominal cavity for deep anesthesia. After fully exposing the heart and aorta through thoracotomy, a 20 ml syringe was inserted into the aorta with an infusion needle from the apex of the heart. The needle was fixed and the right atrium was cut open. 30 ml of physiological saline at 4° C. was perfused through the aorta until most of the blood flowed out. The hippocampus and cortical tissue were carefully and quickly separated and stored at −80° C.for subsequent biochemical analysis. The remaining mice were first perfused with 30 ml of 4° C. physiological saline, followed by 30 ml of pre-cooled 4% paraformaldehyde. Then, the whole brain was removed and fixed with 4% paraformaldehyde for another 48 hours. After dehydration, transparency, paraffin embedding, and sectioning, immunohistochemical analysis was performed.

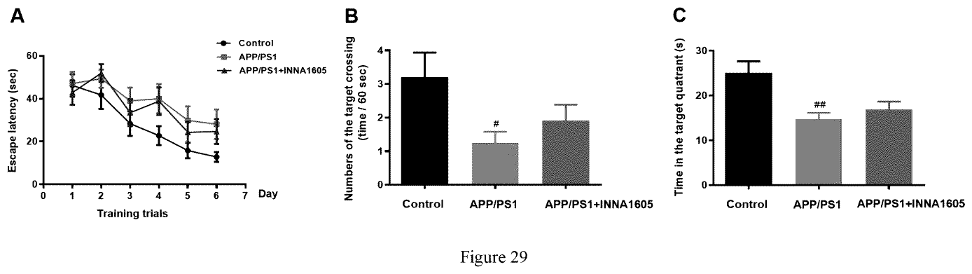

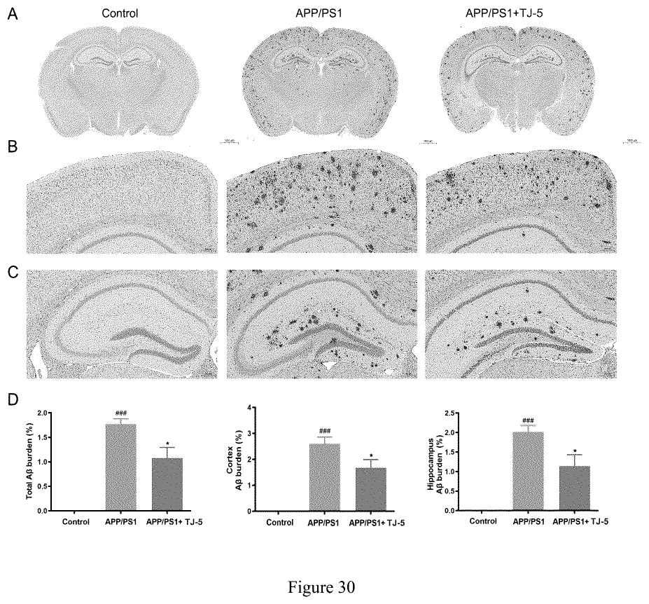

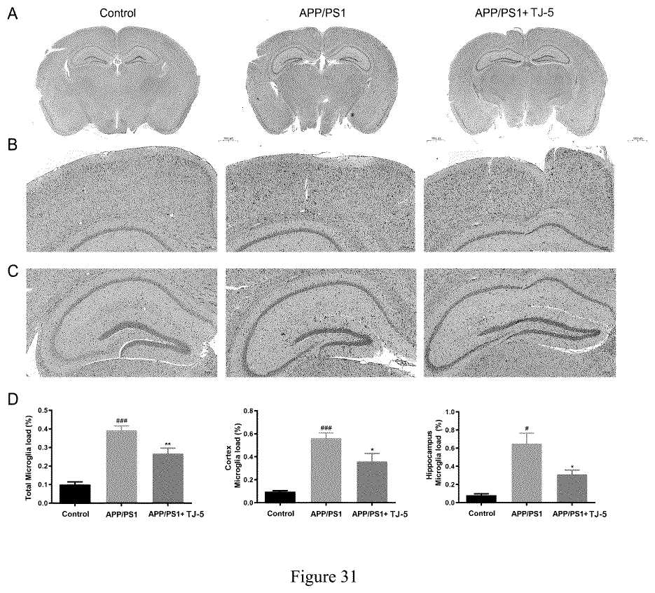

The experimental results obtained are shown in FIG. 29/30/31. The results show that the APP/PSI transgenic mice treated with INNA1605 (TJ-M2010-5, TJ-5) have better memory than APP/PS1 transgenic mice treated with only ddH2O, although it has not yet reached the memory water level of normal mice (i.e. Control group) (see FIG. 29); Through immunohistochemistry, we also found that the APP/PSI transgenic mice treated with INNA1605 had significantly less brain AB deposition in the whole brain (see FIG. 30A), cortex (see FIG. 30B), and hippocampus (see FIG. 30C) regions of the mice compared to the model group, and the Iba-1 positive microglia in the whole brain (see FIG. 31A), cortex (see FIG. 31B), and hippocampus (see FIG. 31C) regions of mice treated with NNA1605 was also significantly reduced compared to the model group. This suggests that INNA1605 can be used as a novel drug for the treatment of Alzheimer's disease, both in terms of efficacy and mechanism.

### Embodiment 13: Aminothiazole Derivative INNA1605 is Used for the Prevention and Treatment of Depression

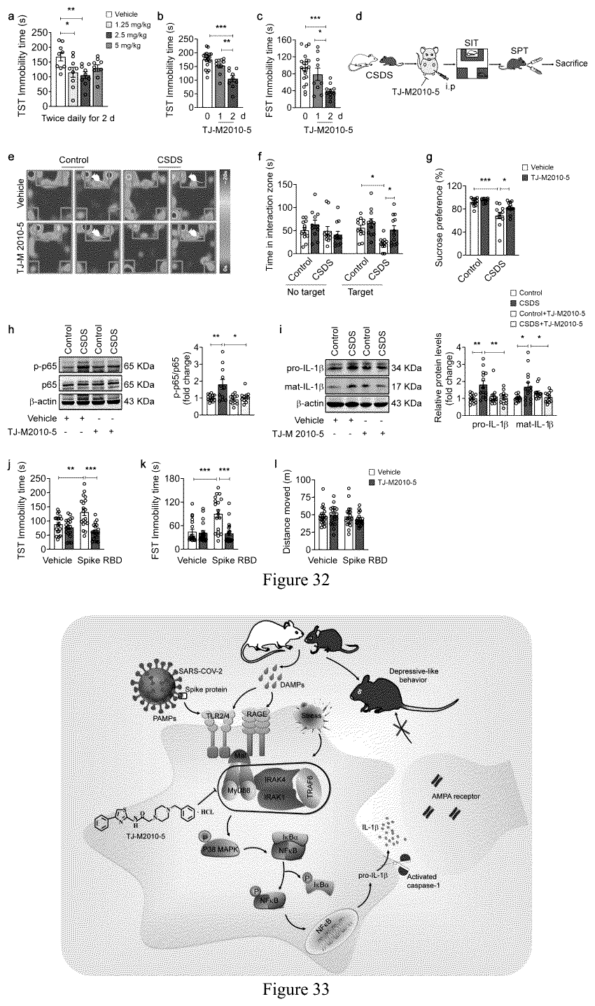

Male C57BL/6J mice (6-7 weeks old, 18-20 g weight) and male CD-1 mice (6-8 years old) were used to create a model of Frustrative Depression (CSDS). C57BL/6J mice were attacked by different CD-1 rats for 10 minutes every day for 10 consecutive days. At night, the two types of mice were kept in the same cage separated by a perforated transparent glass to maintain the pressure of the attack. Experimental results have shown that INNA 1605 (TJ-M2010-5) significantly improves induced depressive behavior and neuroinflammation in the brain (see FIGS. 32 and 33).

### Embodiment 14: Aminothiazole Derivative INNA1605 is Used for the Prevention and Treatment of Transplant Rejection

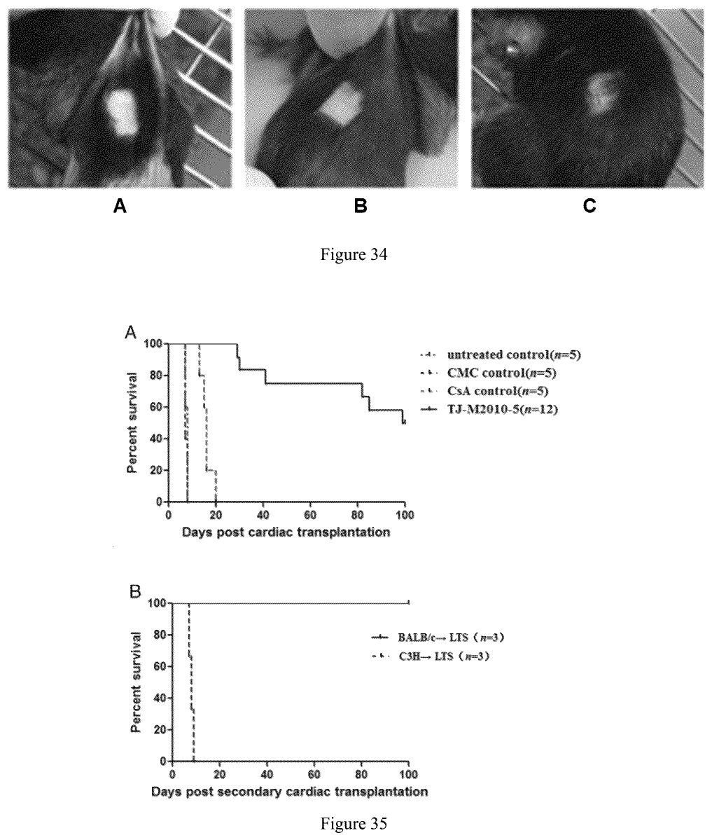

The paired mice for transplantation: The donor mouse is a balb/c mouse; The recipient mouse is B6 mouse. Transplant the donor's skin or heart separately to the recipient. After skin transplantation, INNA1605 (TJ-M2010-5) was continuously used for 2 weeks and then stopped. About 38% of the transplanted skin was permanently non-rejection. After administering TJ-M2010-5 for two weeks after allogeneic heart transplantation, 60% of the heart grafts were permanently not rejected. In these surviving recipient mice (non-rejection), the hearts of the same donor mice were transplanted for a second time without any treatment. The result was 100% non-rejection of the heart, confirming the development of immune tolerance against the same antigen (see FIGS. 34 and 35).

### Embodiment 15: Aminothiazole Derivative INNA1605 is Used for the Prevention and Treatment of Liver Cancer

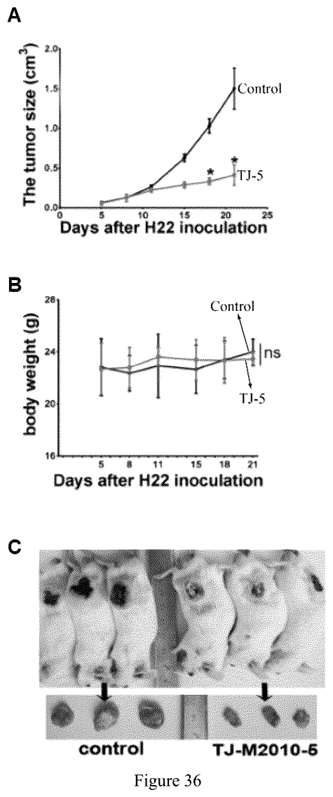

H22 hepatocellular carcinoma cells were subcutaneously implanted in balb/c mice and allowed to grow freely. The animals were divided into two groups: the INNA1605 (TJ-M2010-5) group and the non-drug control group. The tumor size was measured, and it was found that the tumor in the INNA1605 (TJ-M2010-5) group was significantly smaller than that in the control group (see FIG. 36).

### Embodiment 16: Aminothiazole Derivative INNA1605 is Used for the Prevention and Treatment of Obesity and Fatty Liver

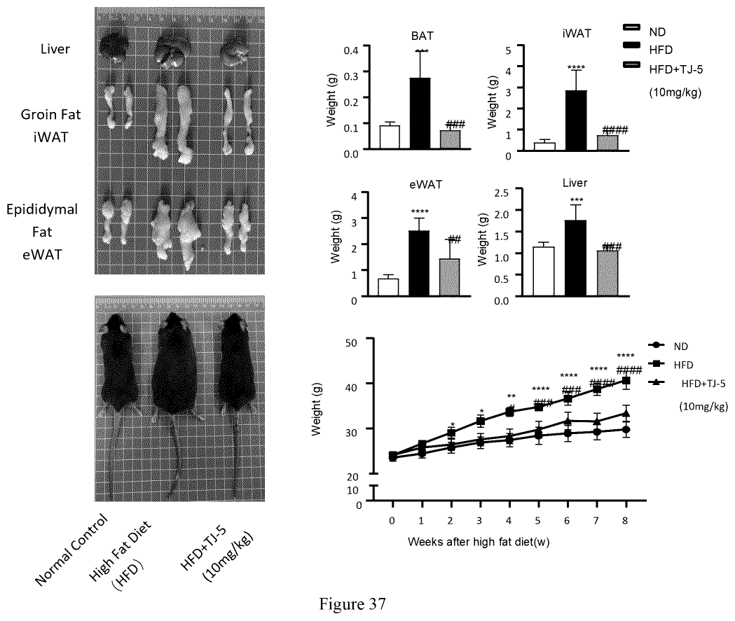

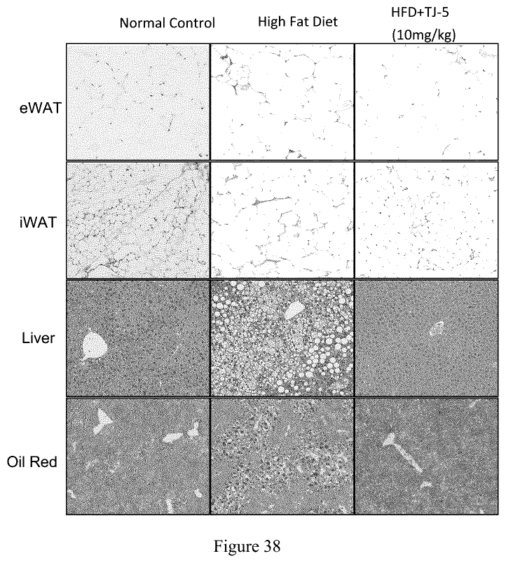

Feed B6 mice with a high-fat diet (HFD) to create an obesity model. Divide the animals into three groups: normal diet group, high-fat diet group, and high-fat diet group with INNA1605 (TJ-M2010-5) added at the same time. Conduct three control groups to measure body weight, inguinal and epididymal fat size, liver size, and examine the liver and various adipose tissues of each group. The results showed that the adipose tissue, liver, and body weight of the group treated with INNA1605 (TJ-M2010-5) were significantly smaller than those of the obese group and close to those of normal mice (see FIG. 37). The pathological examination showed that the lipid droplets in the adipose tissue and liver of the INNA1605 (TJ-M2010-5) group were significantly smaller than those of the obese group, and were similar to normal mice (FIG. 38).

### Embodyment 17: INNA1605 (TJ-M2010-5, TJ-5) Inhibits Suppress Pyroptosis in Mice Model of Hepatic IRI

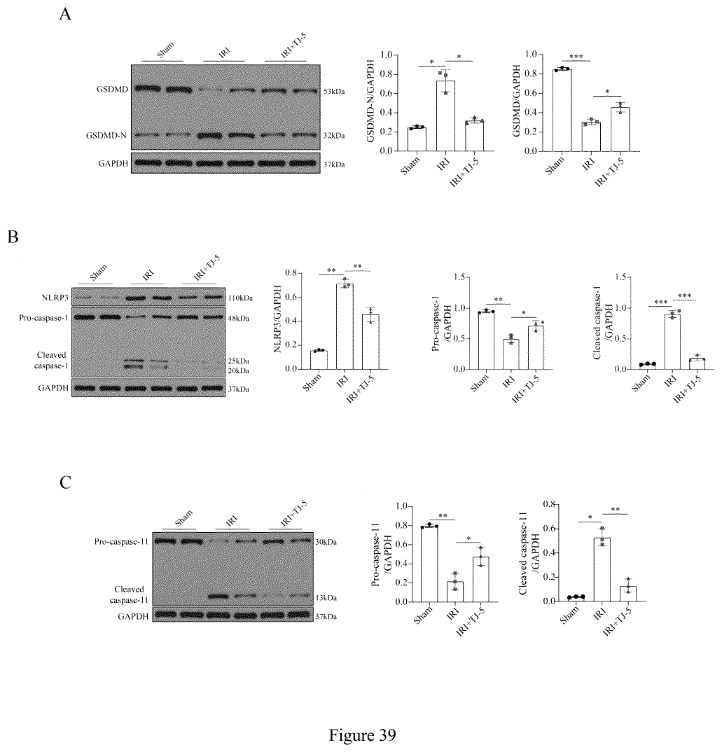

The IRI model of mouse liver was established. Animals were divided into three groups: Sham group, IRI group and IRI+TJ-5 group. Protein extracted from mouse liver was detected. The experimental results show that GSDMD was cleaved to produce more GSDMD-N following hepatic IRI. Interestingly, TJ-5 effectively inhibited GSDMD cleavage. Subsequently, we examined the protein levels of NLRP3 and the cleavage of caspase-1 and found that both were markedly upregulated, whereas those of pro-caspase-1 were downregulated following IRI. However, this trend was reversed following TJ-5 treatment. Furthermore, we focused on the noncanonical pyroptosis pathway and found that TJ-5 effectively inhibited cleavage of caspase-11 caused by ischemia-reperfusion (FIG. 39).

### Embodiment 18: INNA1605 (TJ-5) is Used for the Prevention and Treatment of Dry Eye Disease

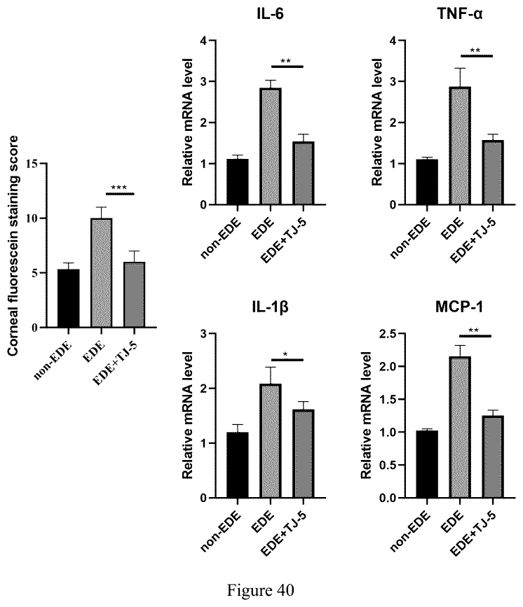

Dry eye disease was induced by placing mice in a experimental dry eye (EDE) condition and administering scopolamine to maximize ocular surface dryness. Animals were divided into three groups: non-EDE group, EDE group and EDE+TJ-5 group. Mice were euthanized on day 9 for cellular and molecular analysis. The results showed that TJ-5 treatment mice displayed significantly lower corneal fluorescein staining score at day 9 and reduced expression of IL-1β, TNF-α, MCP-1 and IL-6 in corneal tissues as compared with EDE group at day 9 (FIG. 40).

### Embodiment 19: INNA1605 (TJ-5) is Used for the Prevention and Treatment of Allergic Rhinitis (AR)

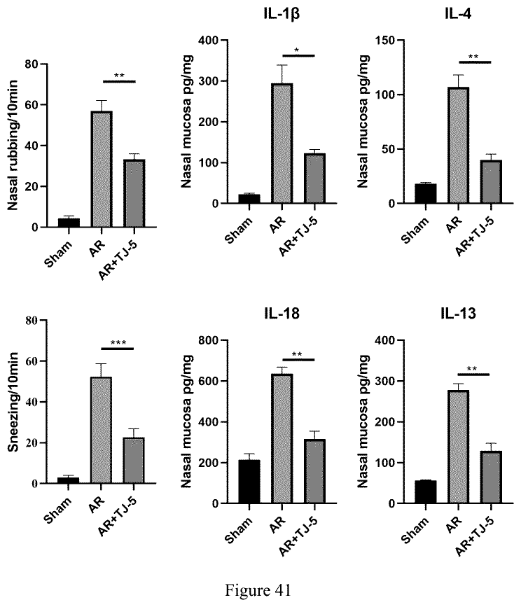

To establish a model of AR, mice were injected intraperitoneally with 25 μg ovalbumin (OVA) and 2 mg aluminum hydroxide on days 0, 7, and 14 to promote primary sensitization. One week after the primary sensitization, the secondary immunization was built. Mice were intranasally challenged with 3% OVA daily from 21 to 35 days. Animals were divided into three groups: Sham group, AR group and AR+TJ-5 group. After 35 days of OVA administration, the number of mice rubbing and sneezing within 10 min was recorded. Nasal lavage fluid was collected 12 h after OVA treatment under anesthesia. The results showed that AR mice suffered from severe sneezing and nasal rubbing compared with sham group, whereas TJ-5 treatment relieved these symptoms. Meanwhile, the levels of IL-4, IL-1β, IL-18 and TNF-α in nasal mucosa were significantly increased in AR group compared with sham group, whereas their levels were decreased by TJ-5 treatment (FIG. 41).

### Embodiment 20: INNA1605 (TJ-5) Contributes to Wound Healing and Reducing Sarring Following Burn or Skin Trauma

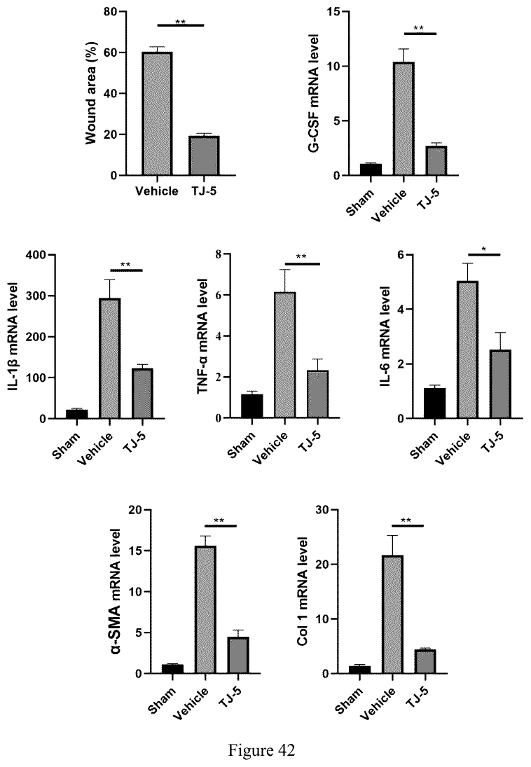

The mice were anesthetized with isoflurane, and 1.5×0.5 cm full-thickness defects were created on their dorsal skin. Animals were divided into three groups: Sham group, Vehicle group and TJ-5 group. After 1 week, mice were sacrificed and wound tissue was harvested for analysis. The results showed that the levels of IL-1β, IL-6, TNF-α, G-CSF, Col I, and α-SMA mRNAs were significantly lower in wound tissue treated with TJ-5 than in untreated. Wounds treated with TJ-5 showed a significant decrease in wound area compared to the vehicle (FIG. 42).

What is claimed is:

1. A method for treating pyroptosis, comprising: administering an effective amount of aminothiazole derivative to cells derived from a human or animal;
the aminothiazole derivative has the following formula: 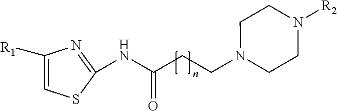
wherein, R1 is phenyl group or substituted aromatic group;
R2 is benzyl, substituted benzyl, phenyl, or substituted phenyl;
n=0, 1, 2.

2. The method of claim 1, wherein the aminothiazole derivative is selected from:
3-(4-benzylpiperazine-1-yl)-N-(4-phenylthiazole-2-yl) propionamide;
3-(4-benzylpiperazine-1-yl)-N-(4-(3-methylphenyl)thiazole-2-yl) propionamide;
3-(4-benzylpiperazine-1-yl)-N-(4-(4-methylphenyl)thiazole-2-yl) propionamide;
3-(4-benzylpiperazine-1-yl)-N-(4-(3-methoxyphenyl)thiazole-2-yl) propionamide;
3-(4-(3-methoxyphenyl) piperazine-1-yl)-N-(4-phenylthiazole-2-yl) propionamide;
3-(4-(4-methoxyphenyl) piperazine-1-yl)-N-(4-phenylthiazole-2-yl) propionamide;
3-(4-(3-methylphenyl) piperazine-1-yl-N-(4-(3-methylphenyl) thiazole-2-yl) propionamide;
2-(4-phenylpiperazine-1-group)-N-(4-phenylthiazole-2-group) acetamide;
2-(4-(4-methylphenyl) piperazine-1-yl)-N-(4-phenylthiazole-2-yl) acetamide;
2-(4-(4-methoxyphenyl) piperazine-1-yl)-N-(4-phenylthiazole-2-yl) acetamide;
2-(4-benzylpiperazine-1-yl)-N-(4-phenylthiazole-2-yl) acetamide;
2-(4-benzylpiperazine-1-yl)-N-(4-(3-methylphenyl)thiazole-2-yl) acetamide;
2-(4-benzylpiperazine-1-group)-N-(4-(4-methylphenyl)thiazole-2-group) acetamide.

3. The method of claim 2, wherein the aminothiazole derivative is administered by intraperitoneal injection.

4. A method for inhibiting activity of NLRP3, Caspase-1, Caspase-11, and Gasdermin D in cells derived from a human or animal, comprising: administering an effective amount of aminothiazole derivative to the cells;
the aminothiazole derivative has the following formula: 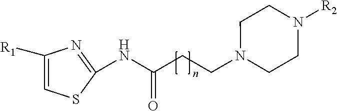
wherein, R1 is phenyl group or substituted aromatic group;
R2 is benzyl, substituted benzyl, phenyl, or substituted phenyl;
n=0, 1, 2.

5. The method of claim 4, wherein the aminothiazole derivative is selected from:
3-(4-benzylpiperazine-1-yl)-N -(4-phenylthiazole-2-yl) propionamide;
3-(4-benzylpiperazine-1-yl)-N -(4-(3-methylphenyl)thiazole-2-yl) propionamide;
3-(4-benzylpiperazine-1-yl)-N-(4-(4-methylphenyl)thiazole-2-yl) propionamide;
3-(4-benzylpiperazine-1-yl)-N-(4-(3-methoxyphenyl)thiazole-2-yl) propionamide;
3-(4-(3-methoxyphenyl) piperazine-1-yl)-N-(4-phenylthiazole-2-yl) propionamide;
3-(4-(4-methoxyphenyl) piperazine-1-yl)-N-(4-phenylthiazole-2-yl) propionamide;
3-(4-(3-methylphenyl) piperazine-1-yl -N-(4-(3-methylphenyl)thiazole-2-yl) propionamide;
2-(4-phenylpiperazine-1-group)-N-(4-phenylthiazole-2-group) acetamide;
2-(4-(4-methylphenyl) piperazine-1-yl)-N-(4-phenylthiazole-2-yl) acetamide;
2-(4-(4-methoxyphenyl) piperazine-1-yl)-N-(4-phenylthiazole-2-yl) acetamide;
2-(4-benzylpiperazine-1-yl)-N-(4-phenylthiazole-2-yl) acetamide;
2-(4-benzylpiperazine-1-yl)-N-(4- (3-methylphenyl)thiazole-2-yl) acetamide;
2-(4-benzylpiperazine-1-group)-N-(4-(4-methylphenyl)thiazole-2-group) acetamide.

6. An external preparation for treatment of superficial chronic inflammation, superficial chronic inflammation occurs on the surface of skin or mucosa and involves a process of pyroptosis, the ointment comprises the following components:
Oil phase: liquid paraffin, stearic acid, white vaseline, octadecyl alcohol;
Aqueous phase: hydroxyphenylate, sodium dodecyl sulfonate, triethanolamine, water, VE;
Essence phase: glycerin, essence, azone;
matrix: stearic acid, lanolin, VE, and octadecyl alcohol.
Drug phase: aminothiazole derivative which has the following formula: 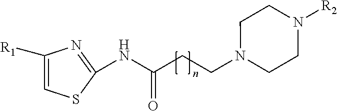
wherein, R1 is phenyl group or substituted aromatic group;
R2 is benzyl, substituted benzyl, phenyl, or substituted phenyl;
n=0, 1, 2.

7. The external preparation of claim 6, wherein the aminothiazole derivative is selected from:
3-(4-benzylpiperazine-1-yl)-N-(4-phenylthiazole-2-yl) propionamide;
3-(4-benzylpiperazine-1-yl)-N-(4-(3-methylphenyl) thiazole-2-yl) propionamide;
3-(4-benzylpiperazine-1-yl)-N-(4-(4-methylphenyl) thiazole-2-yl) propionamide;
3-(4-benzylpiperazine-1-yl)-N-(4-(3-methoxyphenyl) thiazole-2-yl) propionamide;
3-(4-(3-methoxyphenyl) piperazine-1-yl)-N-(4-phenylthiazole-2-yl) propionamide;
3-(4-(4-methoxyphenyl) piperazine-1-yl)-N-(4-phenylthiazole-2-yl) propionamide;
3-(4-(3-methylphenyl) piperazine-1-yl-N-(4-(3-methylphenyl)thiazole-2-yl) propionamide;
2-(4-phenylpiperazine-1-group)-N-(4-phenylthiazole-2-group) acetamide;
2-(4-(4-methylphenyl) piperazine-1-yl)-N-(4-phenylthiazole-2-yl) acetamide;
2-(4-(4-methoxyphenyl) piperazine-1-yl)-N-(4-phenylthiazole-2-yl) acetamide;
2-(4-benzylpiperazine-1-yl)-N-(4-phenylthiazole-2-yl) acetamide;
2-(4-benzylpiperazine-1-yl)-N-(4-(3-methylphenyl)thiazole-2-yl) acetamide;
2-(4-benzylpiperazine-1-group)-N-(4-(4-methylphenyl)thiazole-2-group) acetamide.

8. The external preparation of claim 6, wherein the external preparation is an ointment, a liniment, a spray, a patch, or a drop.

9. A method for treating depression, alzheimer's disease, obesity, fatty liver, burn injury, skin wound, rhinitis, psoriasis and dry eye syndrome, comprising: administering an effective amount of aminothiazole derivative to cells derived from a human or animal;
the aminothiazole derivative has the following formula: 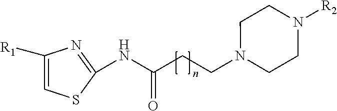
wherein, R1 is phenyl group or substituted aromatic group;
R2 is benzyl, substituted benzyl, phenyl, or substituted phenyl;
n=0, 1, 2.

10. The method of claim 9, wherein the depression is induced by COVID-19 spike protein.
